(19) United States
(12) Patent Application Publication
(10) Pub. No.: US 2025/0354992 A1
(43) Pub. Date: Nov. 20, 2025

(54) MULTIPLEX IMMUNOBLOT EARLY CANCER DIAGNOSTICS TEST FOR VETERINARY DIAGNOSTICS

(71) Applicants: Khristofor Agassandian, Alpharetta, GA (US); Marianna Agassandian, Alpharetta, GA (US)

(72) Inventors: Khristofor Agassandian, Alpharetta, GA (US); Marianna Agassandian, Alpharetta, GA (US)

(21) Appl. No.: 19/197,987

(22) Filed: May 3, 2025

Related U.S. Application Data
(60) Provisional application No. 63/649,324, filed on May 17, 2024.

(51) Int. Cl. G01N 33/574; G01N 21/78

(52) U.S. Cl. CPC G01N 33/57488; G01N 21/78; G01N 33/574; G01N 2474/10

(57) ABSTRACT

The invention provides a qualitative immunoblot-based in vitro method for the detection of onconeural antibodies class IgG to twelve different antigens (amphiphysin, CV2, PNMa2/Ta, Ri, Yo, Hu, recoverin, SOX1, titin, zic4, GAD65 and Tr (DNER)) in serum or plasma samples of mammalian animals such as dogs, cats, ferrets, and rabbits for early diagnosis of twenty two cancer and cancer-associated neurological diseases. Detection of these antibodies in the blood of the animals is confirmed via an indirect immunofluorescent assay. Examples, including enzyme anti-dog, anti-cat, anti-ferret, and anti-rabbit conjugates, and serum or plasma quantities, are provided.

### CROSS-REFERENCE TO RELATED APPLICATIONS

This application claims the benefit of U.S. Provisional Patent Application Ser. No. 63/649,324, filed May 18, 2024, with the same title, the entire content of which is incorporated herein by reference.

### BACKGROUND

The following information is provided to assist the reader in understanding the technologies disclosed below and the environment in which such technologies may typically be used. The terms used herein are not intended to be limited to any particular narrow interpretation unless clearly stated otherwise in this document. References set forth herein may facilitate understanding of the technologies or the background thereof. The disclosure of all references cited herein is incorporated by reference.

Cancer is one of the most common causes of death in dogs, cats, ferrets, and rabbits, and this incidence is 10 times higher in pets compared to humans. According to recent estimations, the US households own an estimated 90 million dogs (canine), 46.5 million cats (feline), over 5 million ferrets (musteline), and 2.2 million rabbits (leporine). These companion animals live in more than 88 million households in Europe. According to the Perseus Foundation, the estimates of cancer incidence indicate that there are around 6 million new cancer detections made in dogs and a comparable number made in cats every year. Thus, approximately 12 million dogs and cats in the United States alone are newly diagnosed with cancer every year. About 50% of ferrets at age 3 and about 80% of unspayed rabbits develop different cancers. Recent statistics have shown that spontaneous malignant tumors can develop in one in four dogs and one in five cats in their lifespans. Despite this, the world still lacks effective early cancer detection techniques and therapies in veterinary medicine for the best outcome for companion animals. Hence, early detection of cancer is an important step in reducing mortality and improving the quality of life for pets and their owners.

Recently, two new advanced techniques were adopted from human cancer diagnostics. The first method of early multi-cancer detection in dog-only test, OncoK9, is based on next-generation sequencing technology (Flory et al., 2022). This “liquid biopsy” test is relatively sensitive and specific. The test requires from fifteen to seventeen milliliters of blood, which is a significant amount and may not be suitable for medium and small dog breeds. Due to the expensive technology, this test was not affordable for pet owners with a general and low income. The test helped to see if a cancer was present, but did not specify the type of cancer, except for the type of lymphoma. Consequently, due to the price and a lack of details about the type of cancer detected, this test went off the veterinary diagnostics market. The second test, coming from human diagnostics and used for early cancer detection, is Nu.Q. This test is based on analyzing and measuring the level of nucleosomes in dog blood (Dolan et al., 2021). However, the elevated levels of nucleosomes in plasma can be associated not only with malignancies but also with severe or systemic inflammation caused by other diseases, trauma, or autoimmune disorders that Nu.Q cannot differentiate from cancers. Another downfall of the Nu.Q test is the inability to differentiate between cancer types, limiting test results to a simple positive or negative determination for only six cancers. Note that the early cancer detection test is still unavailable for cats, ferrets, and rabbits.

The limitations of these two tests illustrate the need to develop a new approach for early cancer detection in pets.

Antibodies are an important clinical tool widely used in human medicine as diagnostic biomarkers for various diseases (Shurin & Wheeler, 2024), in the distinction of veterinary medicine, where the usage of antibody biomarkers is limited due to a lack of development, standardization, and other limitations (O'Kell et al., 2022; Rohdin et al., 2024). In addition, recent studies using constructed cancer models have demonstrated the equivalence of canine-human cancer models and shared cancer biology between canines and humans (Tawa et al., 2021).

Onconeural antibodies are the antibodies that form against some of the neuronal proteins/antigens abnormally expressed in a variety of tumors and normally present in the central and peripheral nervous systems (Chatterjee et al., 2017). Thus, immune responses that are caused by tumors may be misdirected against the nervous system because of protein/antigen identity between tumor cells and healthy neuronal cells. That can lead to tumor-associated Paraneoplastic neurological syndromes (PNS) that precede the manifestations of cancer (Honnorat & Antoine, 2007; Marsili et al., 2023). The detection of onconeural antibodies that can be formed against intracellular or extracellular neuronal proteins is an important tool in the diagnosis and management of these disorders (Chatterjee et al., 2017; Ruiz-García et al., 2020). The antibodies directed against intracellular proteins are detected in most cancer-bearing patients without the development of any neurological diseases and are specific for malignancy rather than for neurological syndrome (Graus et al., 2021; Kannoth, 2012). These antibodies are also termed “high-risk” antibodies since their formation precedes cancer manifestation. Detection of onconeural/high-risk (ONHR) antibodies leads to a search for an underlying malignancy (>85%) (Graus et al., 2021).

Depending on the type of tumor, tumor cells expressing antigens that are normally present in the nervous system such as intracellular amphiphysin, CV2, PNMa2/Ta, Ri, Yo, Hu, recoverin, SOX1, titin, Zic4, GAD65, and Tr (DNER) which can induce the formation of specific ONHR antibodies at a time where cancer is not detected by conventional methods (ultrasound, MRI or X-ray) and is not symptomatic in humans (Chen et al., 2020; D'Alessandro et al., 2010; Darnell et al., 2000; Floyd et al., 1998; Graus et al., 1997, 2004; Kazarian & Laird-Offringa, 2011; Lei et al., 2020; Matsubara et al., 1996; O'Leary et al., 2017; Tan et al., 2014; Wang et al., 2020; Wei et al., 2023). In contrast to intracellular antigens, the antibodies against extracellular antigens such as AMPAR, GABABR, mGluR5, P/Q VGCC, NMDAR, CASPR2, GFAP, LGI1, DPPX, GlyR, AQP4, and MOG are usually more related to neurological disease than cancers (Budhram & Sechi, 2024). When tumor cells start to normally express not antigenic proteins at high levels or alter their localization, or these antigens are derived from normal genes by somatic mutation or single nucleotide polymorphisms (SNPs), epigenetic modifications (glycosylation, phosphorylation, methylation, acetylation, deamidation, and isomerization), chromatin remodeling, or deletions, they can stimulate an immune response due to having a novel sequence (Doyle & Mamula, 2012; Glisovic et al., 2008; Keene, 2007). They can be recognized as non-self by the immune system and promote ONHR antibody formation.

The onconeural antibodies can be detected in the human blood of patients with different types of tumors and have the potential to be an early sign of cancer presence. Most of the ONHR antibodies to intracellular antigens found in humans have not been identified yet in veterinary cohorts, including such mammalian animals as dogs, cats, ferrets, and rabbits, and only a few antibodies, such as LGI1, NMDR, GABAaR to extracellular antigens, and GAD65 were detected in some animals (Binks et al., 2022; Davison et al., 2008; Glantschnigg-Eisl et al., 2023; Hemmeter et al., 2023; O'Kell et al., 2022; Pancotto & Rossmeisl, 2017; Rohdin et al., 2024).

Recent studies, using cancer models, have demonstrated the equivalence of canine-human cancer models and shared cancer biology between canines and humans (Tawa et al., 2021).

The comparative oncology in other companion animals has demonstrated translational relevance to human cancers (Fernández-Fournier et al., 2022; Haukanes et al., 2015; Oh & Cho, 2023; Schiffman & Breen, 2015; Stafford et al., 2019; Tawa et al., 2021). The evaluation of the association of companion animal cancers with human cancers allowed the translation of diagnostic markers to animal oncology, which is a powerful tool for the development of novel methods in early cancer diagnostics and therapy. The comparative sequence analysis revealed a high similarity of intracellular antigens amphiphysin, CV2, PNMa2/Ta, Ri, Yo, Hu, recoverin, SOX1, titin, Zic4, GAD65, and Tr (DNER) between human, dog, cat, ferret, and rabbit species (Table 1 A-D).

An immunoblot assay for the diagnosis of paraneoplastic syndromes in humans was developed several years ago using the detection of ONHR antibodies associated with these syndromes (Dèchelotte et al., 2020; Kurien & Hal Scofield, 2015; Mahmood & Yang, 2012; Sormunen et al., 2023). This method is highly selective with low limits of detection, applicable to the determination of a range of autoantibodies, and inexpensive. Nitrocellulose Blot strips used as antigen-containing solid phase are coated with immobilized recombinant antigens: amphiphysin, CV2, PNMa2/Ta, Ri, Yo, Hu, recoverin, SOX1, titin, Zic4, GAD65, and Tr (DNER). Thus, these multiparameter immunoblots that contain panels with a broad range of recombinant characterized antigens provide efficient multiparameter monospecific autoantibody detection. The correct performance of the individual incubation steps is indicated by the staining of the control band at the lower end of each strip. The evaluation of the results is performed fully automatically (Scharf et al., 2018).

Multiplex indirect immunofluorescence assay is a crucial method for the detection of ONHR autoantibodies. Using BIOCHIP Mosaics composed of different primate tissues, including nerves, cerebellum, pancreas, and intestine, investigation of multiple antibodies can be accomplished simultaneously, allowing complete screening of autoantibodies against known and unknown target antigens (Godelaine et al., 2019; van Beek et al., 2020). Multiple antibodies investigated in parallel using BIOCHIP Mosaics based on the monkey brain section provide a comprehensive autoantibody screening that enables the detection of autoantibodies against different target antigens. This screening includes two steps where specific antibodies from the diluted patient samples bind to associated neuronal proteins in the BIOCHIP in the first step, and then fluorescein (FITC)-labeled secondary antibodies bind to specific primary antibody/antigen complexes from patient samples. The complexes can be visualized by excitation with respective wavelengths using a fluorescent microscope.

### SUMMARY

The present invention builds upon the unanticipated finding that, within the field of veterinary diagnostics, the presence of onconeural antibodies demonstrates a significantly stronger correlation with malignant neoplasms than with primary neurological disorders. This insight diverges from the conventional paradigm, which primarily associates onconeural antibodies with paraneoplastic neurological syndromes in human medicine. By leveraging this novel association, the invention enables the novel repurposing of existing diagnostic assays, originally designed for the detection of human neurological disorders, to identify onconeural/high-risk (ONHR) antibodies in mammalian animals. This cross-species application facilitates a transformative approach for the early detection of malignancies in veterinary contexts, providing a sensitive and non-invasive biomarker-based method to screen for underlying cancers. The diagnostic strategy holds particular promise for enhancing prognostic outcomes through earlier therapeutic intervention, thereby bridging a critical gap in comparative oncology and translational diagnostic methodologies.

The invention provides the first and only immunoblot-based and immunofluorescent staining-based ONHR antibody detection methods for the early diagnosis of cancers in companion mammalian animals such as dogs, cats, ferrets, and rabbits. The anti-amphiphisyn, -CV2, -PNMa2/Ta, -Ri, -Yo, -Hu, -recoverin, -SOX1, -itin, -Zic4, -GAD65 and -Tr (DNER) antibodies against intracellular neuronal antigens associated with specific types of tumors can be detected in the serum or plasma samples of mammalian animals to diagnose suspicious underlining cancers. These ONHR antibodies can be used to screen for a variety of different types of cancers in animals during their wellness exam (a preventative screening), when a veterinarian suspects a cancer, or when an animal is genetically predisposed to cancer. Some useful examples of such cancers include basal cell carcinoma, bladder tumor, brain tumor, esophageal cancer, gallbladder tumor, intestinal tumors, kidney tumor, lung cancer, lymphoma, mammary tumors, melanoma, multiple myeloma, neuroblastoma, ovarian tumors, prostate tumor, salivary gland adenocarcinoma, squamous cell carcinoma, testicular tumor, thymoma, thyroid cancer, and uterine tumors, although the invention is not limited in this regard.

The present invention also introduces the first and only method for monitoring cancer status and evaluating treatment efficacy in animals that have undergone surgical or therapeutic interventions. This approach offers reassurance to pet owners and veterinary professionals by enabling an objective assessment of treatment outcomes. The Early Cancer Diagnostics test functions as a valuable follow-up tool in post-treatment care, ensuring continued surveillance for disease recurrence or progression.

A central innovation of this invention is its capacity to detect cancers in companion animals at a very early stage—months or even years before clinical symptoms emerge. This early detection capability facilitates timely therapeutic intervention, significantly improving the likelihood of successful treatment and reducing the risk of advanced disease development. This feature is particularly critical in cancer diagnostics for companion animals, where early signs are often subtle or absent.

Another significant advantage of the invention is its potential as a preventative cancer screening tool for a broad range of mammalian animals. This is especially relevant for breeds known to be genetically predisposed to cancer. In dogs, these include breeds such as Beagle, Bernese Mountain Dog, Boxer, Flat-Coated Retriever, French Bulldog, German Shepherd, Golden Retriever, Great Dane, Irish Wolfhound, Labrador Retriever, Mastiff, Miniature Schnauzer, Pembroke Welsh Corgi, Rhodesian Ridgeback, Rottweiler, Scottish Deerhound, and Siberian Husky. In cats, predisposed breeds include Persian, Bengal, Siamese, Abyssinian, Himalayan, Exotic Shorthair, and Sphynx.

A further technical advantage of this invention is its minimal sample volume requirement. The test can be conducted using only 0.1 to 0.5 milliliters of serum or plasma, making it highly suitable for small and medium-sized breeds as well as other small mammalian species. This eliminates the need for large-volume blood collection, which is especially beneficial for animals with limited blood volume. In contrast, existing cancer diagnostic tests, such as the Nu.Q test and OncoK9 test, require significantly larger volumes of blood (7 ml and 14-17 ml, respectively) and are currently validated only for dogs. The present invention thus expands the accessibility and utility of cancer diagnostics across a broader range of species and clinical scenarios.

An additional advantage of the present invention is that it represents the first diagnostic test in veterinary medicine capable of not only detecting but also differentiating between 22 types of cancer and 16 neurological disorders in animals. When clinical signs raise suspicion of cancer, the Early Cancer Diagnostics test serves as a valuable confirmatory tool to support the veterinarian's diagnosis. Moreover, the test delivers high-quality diagnostic information at an affordable cost, making it accessible to a broad range of animal owners.

Another important feature of the invention is its ability to monitor the effectiveness of cancer treatment in dogs, cats, ferrets, and rabbits. By comparing the presence and levels of ONHR antibodies in serum samples collected before treatment initiation with those obtained at regular intervals during therapy, veterinarians can assess the treatment response with greater precision. In contrast to existing diagnostic tests, which are limited to dogs and lack post-treatment monitoring capabilities, this test offers a comprehensive approach to both diagnosis and ongoing management.

Furthermore, the invention provides critical insight into the relationship between malignancy and paraneoplastic neurological syndromes (PNS). In cases where an animal presents with neurological symptoms such as seizures, the Early Cancer Diagnostics test can help determine whether an underlying cancer is the causative factor. This capability is essential for guiding timely and targeted intervention, enabling veterinarians to uncover and address malignancy-associated neurological manifestations that might otherwise remain undetected.

### DESCRIPTION OF THE DRAWINGS

FIG. 1 is Table 1A showing a human and canine protein sequence alignment.

FIG. 2 is Table 1B, which shows a human and feline protein sequence alignment.

FIG. 3 is Table 1C, which shows a human and ferret protein sequence alignment.

FIG. 4 is Table 1D showing a human and rabbit protein sequence alignment.

FIG. 5 shows exemplary test strips with the immunoblot-based detection of onconeural antibodies in the sera obtained from dogs diagnosed with different types of cancer and healthy dogs.

FIG. 6 contains examples of chart results (panels A, B, and C) corresponding to onconeural antibody detection by immunoblot-based assay in cancer-diagnosed dogs.

FIG. 7 contains further examples in panels D, E, and F.

FIG. 8 contains yet further examples in panels G, H, and I.

FIG. 9 contains further examples in panels J and K. Panel L represents a comparison for a healthy dog.

FIG. 10 contains a further example of a dog with cancer (panel M) and a healthy dog comparison (panel N), with explanations for panels A through N shown in panel O.

FIGS. 11 and 12 illustrate immunofluorescent detection of ONHR antibodies in the same serums used for the results represented in FIGS. 6-9 and collected from cancer-diagnosed (A-J) and cancer-free (healthy, K, L) dogs.

FIGS. 13 and 14 contain an explanation of the results for the Early Cancer Diagnostics test, indicating correlations of detected ONHR antibodies with cancers and PNSs.

FIG. 15 is an exemplary protocol 500 for an Early Cancer Diagnostic test based on Immunoblot onconeural antibody detection.

FIG. 16 is an exemplary protocol 600 for an Early Cancer Diagnostic test based on the immunofluorescent onconeural antibody detection.

FIGS. 17 and 18 illustrate examples of onconeural antibody detection by immunoblot assay: in the cancer-diagnosed dog (panels A, B): panel A—single onconeural antibody detection by immunoblot assay in the cancer-diagnosed dog, panel B—multiple onconeural antibody detection by immunoblot assay in the cancer-diagnosed dog; in a dog with cancer suspicion (panel C); in a breed-predisposed dog (panel D); example of dog's cancer treatment monitoring by immunoblot assay (panels E, F).

FIGS. 19 and 20 show examples of onconeural antibody detection by immunoblot assay: in the cancer-diagnosed cat (panel A, B): panel A—single onconeural antibody detection by immunoblot assay in the cancer-diagnosed cat, panel B—multiple onconeural antibody detection by immunoblot assay in the cancer-diagnosed cat; in a cat with cancer suspicion (panel C); in a breed-predisposed cat (panel D); example of monitoring the state of cancer diagnosed in the cat by immunoblot assay (panels E, F).

FIGS. 21 and 22 are examples of onconeural antibody detection by immunoblot assay: in the cancer-diagnosed ferret (panel A); in a ferret with cancer suspicion (panel B); in a presumably cancer-free ferret (panel C); in a cancer-free healthy ferret (panel D).

FIGS. 23 and 24 show examples of onconeural antibody detection by immunoblot assay: in the cancer-diagnosed rabbit (panel A); in a rabbit with cancer suspicion (panel B); in a presumably cancer-free rabbit (panel C); in a cancer-free healthy rabbit (panel D).

FIG. 25 shows exemplary onconeural antibody detection test results and test performance for dogs (panel A), cats (panel B), ferrets (panel C), and rabbits (panel D).

### DETAILED DESCRIPTION OF THE INVENTION

The present invention provides methods for detecting onconeural/high-risk (ONHR) antibodies that target intracellular neuronal antigens—such as amphiphysin, CV2, PNMa2/Ta, Ri, Yo, Hu, recoverin, SOX1, titin, Zic4, GAD65, and Tr (DNER), in serum or plasma collected from animals. These antibodies serve as biomarkers for cancer or paraneoplastic neurological syndromes (PNS) triggered by malignancy.

In certain embodiments, the method involves the use of a nitrocellulose membrane test strip embedded with immobilized recombinant forms of these antigens. When serum or plasma from the test subject is applied under specific binding conditions, the presence of ONHR antibodies, if any, leads to the formation of antigen-antibody complexes. The detection of such complexes indicates a high likelihood that the subject animal has cancer or a PNS associated with malignancy.

The method also includes the application of secondary antibodies specific to the immunoglobulin G (IgG) of various species, such as anti-dog IgG, anti-cat IgG, anti-ferret IgG, and anti-rabbit IgG, capable of recognizing both the heavy and light chains of the IgG molecule. These secondary antibodies are conjugated with alkaline phosphatase (AP) to enable colorimetric detection of the antigen-ONHR antibody complexes formed on the test strip. Indirect detection through this mechanism enhances specificity and allows for clear visualization of positive results.

Additionally, the method provides optimized recommendations for the concentration of the AP-conjugated secondary antibodies, established during the course of experimental development, as well as guidance on the appropriate volume and dilution of serum or plasma required for effective detection.

Furthermore, the invention includes protocols for indirect immunofluorescence testing using BIOCHIP slides containing monkey tissue substrates, such as nerve, cerebellum, intestine, and pancreas, to complement the strip-based assay. These procedures offer a robust, multisystem approach to detecting ONHR antibodies and thereby identifying underlying cancer or cancer-induced neurological syndromes in companion and small mammals.

In embodiments, the novel methods include the BIOCHIP containing monkey neuronal tissue, the cerebellum and pancreas tissues with expressed in these tissues selected antigenic proteins, and to bind, if any, to antibodies in serum or plasma samples from dog, cat, ferret, and rabbit subjects.

The antigen/ONHR antibody complex is detected by labeled with fluorescein isothiocyanate (FITC), specific secondary anti-dog IgG, anti-cat IgG, anti-ferret IgG, and anti-rabbit IgG antibodies that are also provided by the present invention. This is an indirect immunofluorescence test (IIFT) where FITC is a fluorescent probe that is used to label antibodies.

The invention provides the detection of ONHR antibodies during the investigation of specific concentrations of serum and plasma used for the IIFT with BIOCHIPs.

The invention also provides anti-dog IgG, anti-cat IgG, anti-ferret IgG, and anti-rabbit IgG secondary antibodies conjugated with fluorescein isothiocyanate (FITC) with recommended concentration developed during the investigation.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting.

All publications, sequences, database entries, and other references mentioned herein are incorporated by reference in their entirety. Other features and advantages of the invention will be apparent from the following detailed description and figures, and from the claims.

All methods and materials similar or equivalent to those described herein can be used in the practice or testing of the present invention, with suitable methods and materials being described herein. All publications, patent applications, patents, and other references mentioned herein are incorporated references in their entirety.

The present disclosure provides a method for diagnosing and differentiating between twenty-two types of cancer in serum or plasma samples collected from veterinary subjects, such as mammalian animals, by using an ONHR antibody to cancer-specific antigens complex. Antigen immobilized on a solid carrier (test strip) may be provided.

Based on recent studies that have demonstrated the equivalence of canine-human cancer models and shared cancer biology between canines and humans, the sequence alignment for comparison of human, canine, feline, musteline, and leporine neural intracellular antigenic proteins was performed.

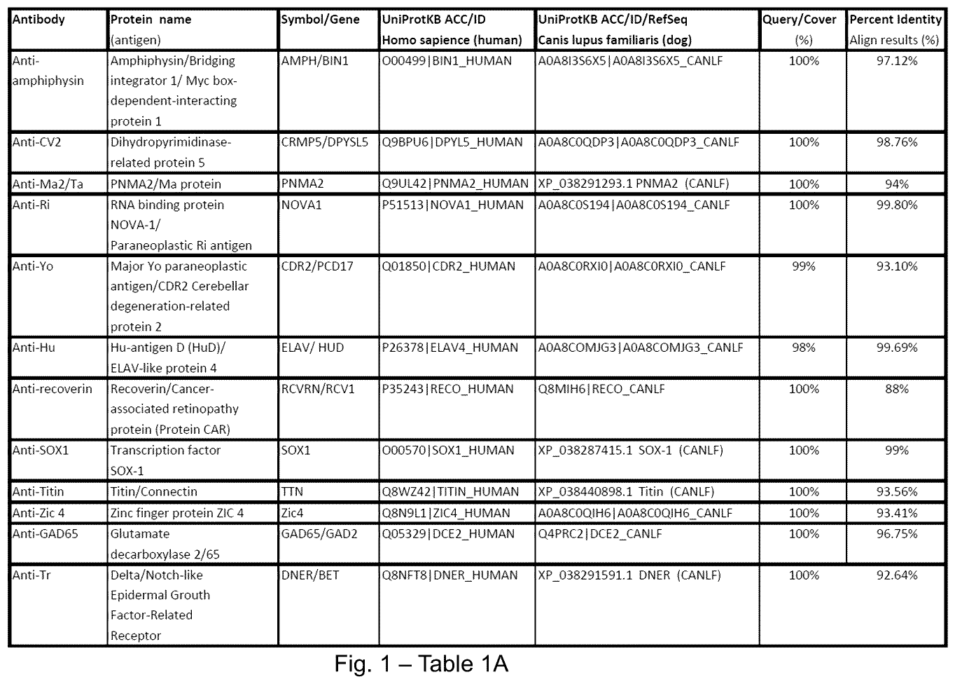

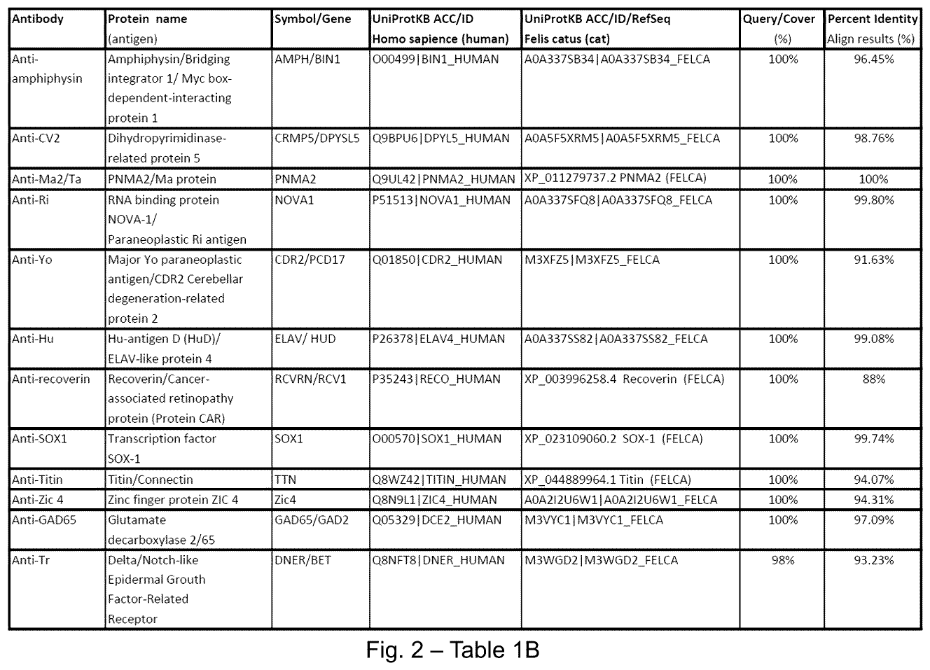

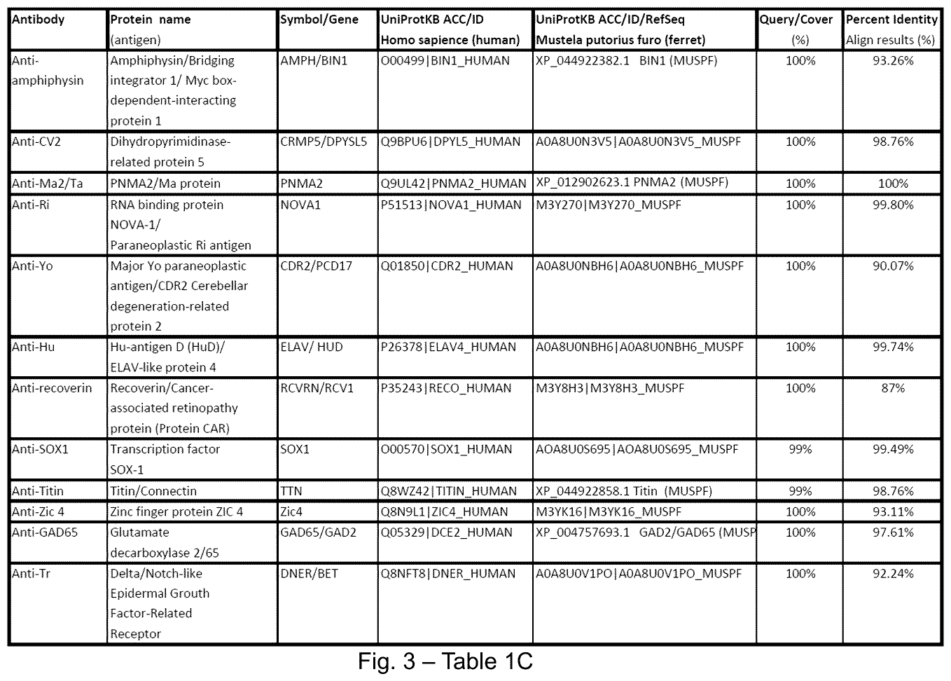

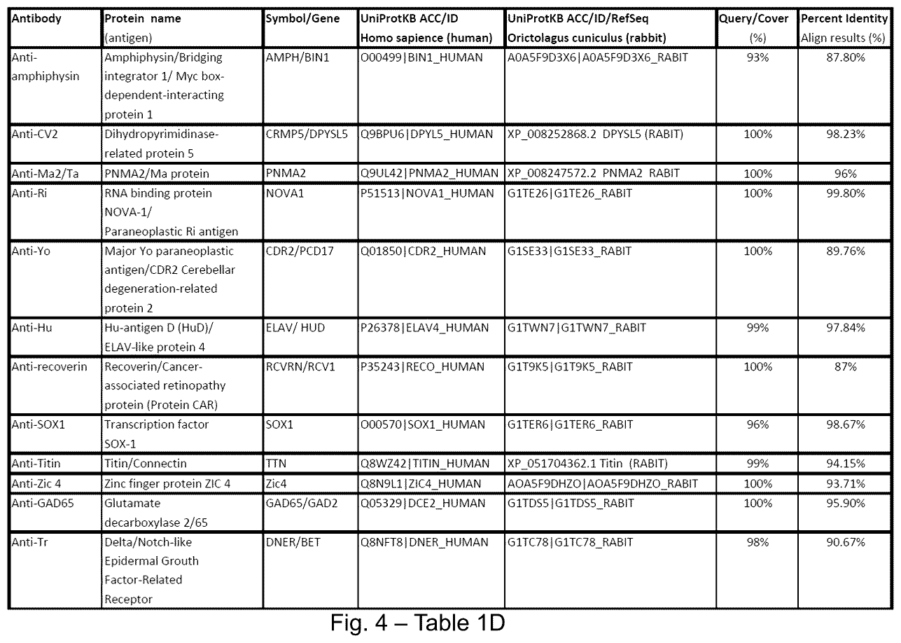

UniProt Knowledgebase (UniProtKB) has been used for the analysis of the following human, canine, feline, musteline, and leporine protein sequences. Human and animal protein sequences were aligned using Clustal Multiple Sequence Alignment. The amphiphysin, CV2, PNMa2/Ta, Ri, Yo, Hu, recoverin, SOX1, titin, Zic4, GAD65 and Tr (DNER) antigens shared significant sequence similarity (from 88% for recoverin to more than 99%) between the human, canine, feline, ferret, and rabbit homologs. FIGS. 1-4 with Table 1 (A-D) are used for illustration.

The method of the present invention comprises the following general steps:
(a) retrieving a blood specimen from the animal,
(b) adding a buffer to the specimen to form a composite specimen,
(c) binding the composite specimen to at least one antigen selected from a group of antigens consisting of amphiphysin, CV2, PNMa2/Ta, Ri, Yo, Hu, recoverin, SOX1, titin, zic4, GAD65, and Tr (DNER), wherein the at least one antigen of the group of antigens is immobilized on a test strip, and
(d) optically detecting a presence of at least one antigen-antibody binding complex using a predetermined threshold.

Test strips containing one, several, or all of the antigens listed above immobilized thereon may be used for veterinary diagnostics to detect and differentiate 22 cancers by using serum or plasma samples obtained from mammalian animals (dogs, cats, ferrets and rabbits) and using certain secondary AP-conjugated antibodies—anti-dog IgG, anti-cat IgG, anti-ferret IgG, and anti-rabbit immunoglobulin class G (IgG) antibodies.

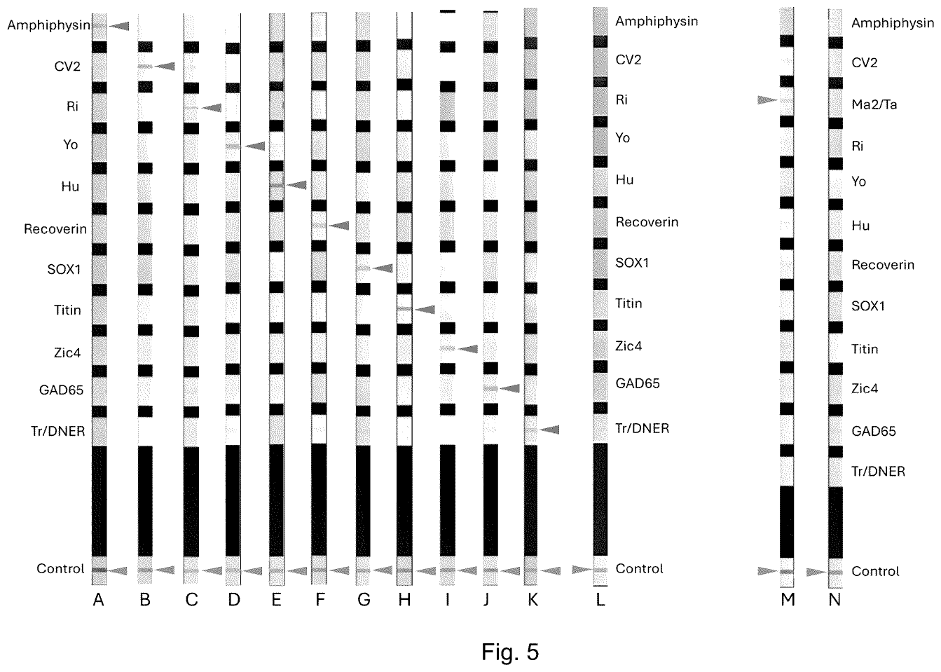

Exemplary performance of the individual incubation steps is indicated by the staining of the control band at the lower end of each strip. This is illustrated in FIG. 5, which shows exemplary test strips with the immunoblot-based detection of onconeural antibodies in the sera obtained from dogs diagnosed with different types of cancer and healthy dogs.

Examples of ONHR antibodies (FIG. 5: anti-amphiphysin (A), -CV2 (B), -Ri (C), -Yo (D), -Hu (E), -recoverin (F), -SOX1 (G), -titin (H), -Zic4 (1), -GAD65 (J), and -Tr/DNER (K), -PNMa2/Ta (M)) bound to associated antigens immobilized on the nitrocellulose strips detected in the serums of cancer-diagnosed dogs. Arrowheads indicate the bands linked to these antibodies and to the control bands, indicating that the experiment was successful. No ONHR antibodies have been detected in the serum of cancer-free (healthy) dogs (L, N). Note that similar results were obtained after the immuno-blot analysis of serums collected from cancer-diagnosed and cancer-free cats, ferrets and rabbits. Strips A-L were used from the EUROLINE Neuronal Antigens Profile 72 (IgG) kit (Code DL 1111-16-01-72 G); and strips M-N from EUROLINE Paraneoplastic Neurologic Syndromes 12 Ag (IgG) kit (Code DL 1111-1601-7G).

If the sample contains specific ONHR antibodies, these bind to the corresponding membrane-bound antigens. In the next step, an alkaline phosphatase (AP) labeled secondary antibody (conjugate) is added, which binds to specific antibodies/membrane-bound antigens complex. The AP catalyzes a colored reaction with the subsequently added nitroblue tetrazolium chloride/5-bromo-4chloro-3-indoyl phosphate (NBT/BCIP). If specific antibodies are present in the patient sample, a dark line appears at the respective antigen position. The evaluation may be automatically performed by using a suitable software package.

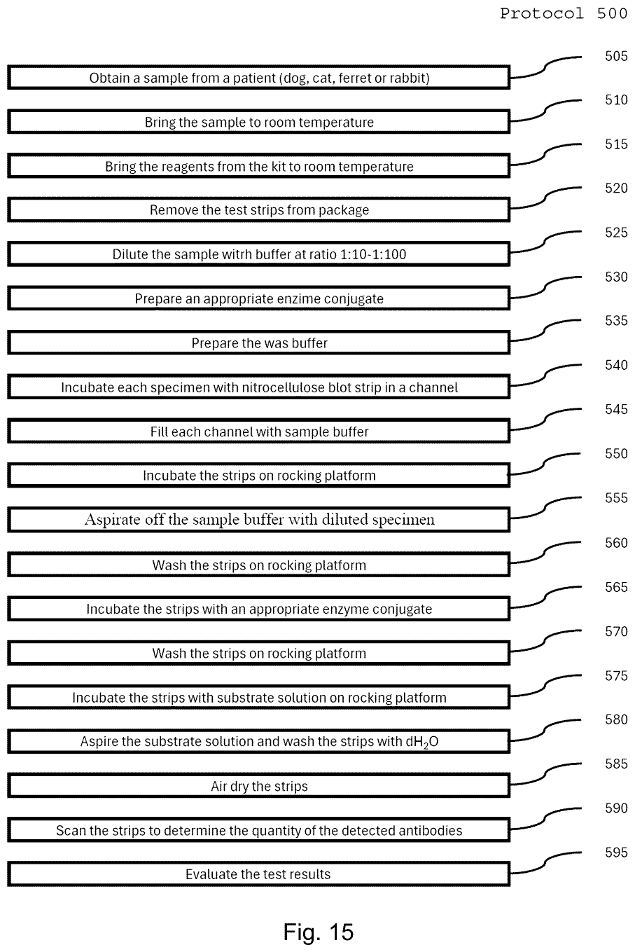

The protocol 500 for ONHR antibody detection in animal serum or plasma using immunoblot assay is seen in FIG. 15. A specimen (also referred to herein as “samples”) obtained from a companion animal can be stored in a refrigerator until use. In step 505, obtain a sample from a subject. In step 510, a serum sample (if refrigerated) should be brought to room temperature (between +18° C. and +25° C.) approximately 30 minutes before use. In step 515, the reagents from the kits should be brought to room temperature (between +18° C. and +25° C.) approximately 30 minutes before use. In Step 520, a bag containing test strips coated with recombinant antigens is opened, and test strips are removed when the room temperature has been reached to prevent condensation. In step 525, the sample (serum or plasma) is diluted at a ratio of 1:10-1:101 in sample buffer containing Tris-buffer saline (TBS-T), 0.1% Tween to reduce background staining, and 2% PBS to block nonspecific binding; and mixed using a vortex. In step 530, enzyme conjugates, which are anti-dog, anti-cat, anti-ferret, and anti-rabbit IgG AP-conjugated, should be prepared by removing the required amount from the bottle with a clean pipette tip and diluting it with the sample buffer according to the assay development, ranging 1:500-1:4000 for anti-dog IgG, 1:100-1:10000 for anti-cat IgG, 1:200-1:2000 for anti-ferret and anti-rabbit IgG. In step 535, a wash buffer, containing TBS-T (preferably provided as a 10× concentrate, should be prepared by diluting the required amount with distilled water at a ratio of 1:10 and 2% of BSA. The ready-to-use wash buffer should be used on the same working day.

In step 540, an incubation of each specimen is performed in the incubation tray with each channel accommodating at least 1.5 ml of solution. The number of test strips, depending on the number of serum samples tested, is placed in empty channels (one strip in one channel). In step 545, each channel is filled with a sample buffer (1.5 ml/channel). In step 550, Incubation with samples occurs by filling each channel with 1.5 ml of diluted serum or plasma in the sample buffer 1:10-1:101 and incubating for 30-90 minutes at room temperature on the rocking platform. The strips are incubated for 5 minutes at room temperature on a rocking platform. In step 555, aspirate off the sample buffer with the diluted specimen. In step 560, wash the strips 3 times for 5 minutes with working strength wash buffer (1.5 ml/channel) on the rocking platform. In step 565, binding to antigen-antibody complexes occurs by adding 1.5 ml of the diluted enzyme conjugate (as mentioned above, step 530) to each channel and incubating for 30-90 minutes on a rocking platform. In step 570, the wash 560 is repeated one time. In step 575, the samples are incubated in 1.5 ml substrate solution, such as Nitroblue tetrazolium chloride/5-Bromo-4-chloro-3-indolylphosphate, NBT/BCIP, (1.5 ml/channel), to visualize the antigen/ONHR antibody bands that represent antigen/ONHR antibody complexes for 10 minutes on a rocking platform.

In step 580, the aspiration of the substrate solution occurs, and the strips are washed with distilled water 3 times in 1 minute. In step 585, the test strips are air-dried. In step 590, the air-dried test strips are scanned with a light scanner and evaluated using a computer to determine the quantity of the detected ONHR antibodies, if any. If specific antibodies are present in the patient sample, a dark line appears at the respective antigen position.

In step 595, the evaluation of test results is automatically performed using suitable optical scanner software. The intensity of the resulting staining, visualized as a dark line on the test strip, is directly proportional to the concentration of ONHR antibodies present in the sample. To interpret the results, a predetermined threshold is applied to the measured band intensity values. In some embodiments, the predetermined threshold is designed to separate a negative result from a positive result: when the intensity of staining is below the predetermined threshold, a conclusion of no cancer present is reached. In other embodiments, the predetermined threshold may be used in a more nuanced manner. Specifically for the examples illustrated herein, band intensity values of 10 or less are considered negative, values between 11 and 25 are classified as low positive, values between 26 and 50 are categorized as positive, and values greater than 50 are designated as strong positive. In this case, values categorized as negative and low positive may be combined and interpreted as no cancer present, as borderline values are still not enough to diagnose cancer with acceptable certainty.

As discussed above, the predetermined threshold may be carefully established to reliably separate a negative result from a positive result and/or to further distinguish varying degrees of antibody presence. Importantly, the threshold values also depend on the specific manner in which the test is performed, including factors such as the type of scanner, sensitivity settings, antigen immobilization density, and the reaction time used. As such, the threshold can be adjusted to accommodate individual test conditions, ensuring optimal accuracy and reproducibility across different laboratory environments and assay setups. This adaptability allows the method to maintain consistent diagnostic performance even when minor procedural variations are present.

Tissue sections of monkey cerebellum, nerve, pancreas, and intestine allow complete screening of antibodies against known and unknown target antigens (Li et al., 2023). Using BIOCHIP Mosaics, composed of different tissues, mentioned above, can be accomplished simultaneously (Arunprasath et al., 2020; van Beek et al., 2020). The BIOCHIP Mosaics, based on the monkey tissue sections, provides a comprehensive autoantibody screening that enables the detection of antibodies against unidentified target antigens expressed in these tissues.

This screening includes two steps where specific antibodies, if any, from the diluted patient samples bind to antigens expressed in the monkey cerebellum, nerve, pancreas, and intestine tissues mounted on the BIOCHIP on the solid-phase, BIOCHIP in the first step, and then fluorescein FITC-labeled secondary anti-dog, anti-cat, anti-ferret, or anti-rabbit IgG antibodies bind to specific ONHR antibody/antigen complex.

The antibody/antigen complex can be visualized by excitation with respective wavelengths in the fluorescent microscope. For the disclosed protocol, the incubation time, dilutions of serums (from 1:5 to 1:100), secondary specific anti-dog, anti-cat, anti-ferret, and anti-rabbit IgG antibodies, and their concentrations (from 1:1500 to 1:10000) have been developed and significantly optimized for the IIFT detection method of ONHR antibodies in dogs', cats', ferrets', and rabbits' serum or plasma.

The following exemplary protocol for ONHR antibody detection by IIFT may be used with serum or plasma samples obtained from mammalian animals, and using different secondary FITC-conjugated anti-dog IgG (SouthernBiotech, Birmingham, AL, USA), anti-cat IgG (Jackson ImmunoResearch Laboratories, West Grove, PA, USA), anti-ferret IgG (Abcam, Waltham, MA, USA), and anti-rabbit IgG (SouthernBiotech, Birmingham, AL, USA) antibodies to detect the complexes of antigens in tissue sections with specific ONHR antibodies in the serum.

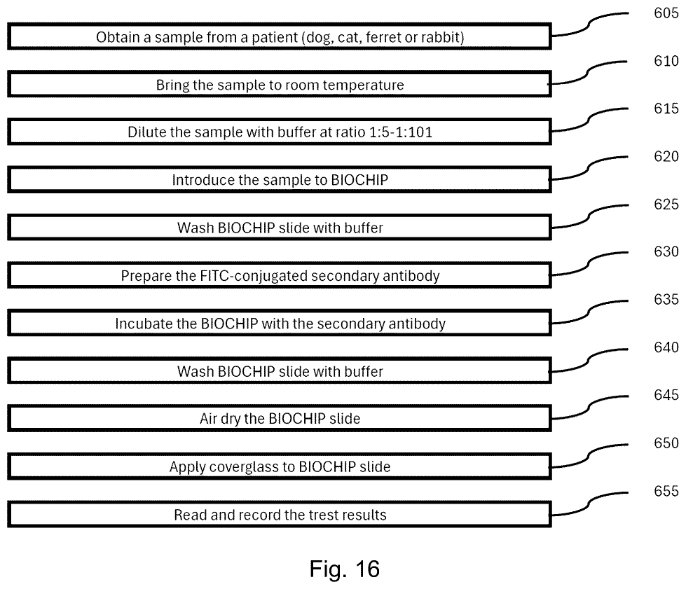

FIG. 16 shows protocol 600 for antibody detection by IIFT in an animal serum using qualitative or semiquantitative in vitro determination of animal ONHR antibodies of immunoglobulin class IgG against neural antigens in patient samples using slides with BIOCHIP (provided by EUROIMMUNE) containing monkey cerebellum, nerve, pancreas, and intestine tissues to identify the presence of ONHR antibodies and support the diagnosis of different cancers. Any slides mounted with the cryosections of tissues that express neuronal antigens can be used for the assay. The person skilled in the art is familiar with methods described in Kyuseok I., Mareninov et al. (Im et al., 2019).

The BIOCHIP (EUROIMMUN, Lubeck, Germany) method combines the screening of antibodies and target antigen-specific substrates in a single incubation field that contains a mosaic of four different substrates, such as a frozen section of monkey cerebellum, nerve, pancreas, and intestine tissues, as mentioned above. The protocol 600 for ONHR antibody detection in animal serum using the indirect immunofluorescent test (IIFT) is shown in FIG. 16. A specimen (also referred to herein as “samples”) obtained from a companion animal can be stored in a refrigerator until use.

In step 605, the method teaches to obtain a sample from a subject animal. In step 610, a serum sample (if refrigerated) should be brought to room temperature (between +18° C. and +25° C.) approximately 30 minutes before use. In step 615, the samples (serum or plasma) were first diluted in PBS-Tween buffer according to the experimental selection via serial dilutions to choose the concentrations suitable for the best results. Thus, the recommended sample dilution/concentrations for the determination of immunoglobulin class IgG antibodies described above are from 1:5 to 1:101. In step 620, the diluted sample is incubated in contact with the cerebellum, pancreas, and intestine tissues immobilized on the slide with a mosaic of BIOCHIPs for 30 minutes at room temperature. (Slides are provided by EUROIMMUN together with cover glasses and are ready to use. The slides must be broad to room temperature before use. The substrates used on the slides are the cerebellum, optic nerve, intestinal tissue, and pancreas. At-home-prepared slides with immobilized frozen sections of the cerebellum, intestinal tissue, or pancreas tissue are also suitable for the assays. If the ONHR antibodies are present in the sample, they are attached to the associated antigens expressed in these tissues.

In step 625, the slides with attached antibodies, if any, are washed 3 times with PBS-Tween. In step 630, the anti-dog, anti-cat, anti-ferret, and anti-rabbit IgG FITC-conjugated IgG antibodies were diluted in PBS-Tween buffer according to the experimental selection of the concentrations suitable for the best results. Recommended sample dilutions/concentrations for semiquantitative evaluation are for anti-dog IgG 1:400-1:4000, anti-cat IgG 1:100-1:10000, anti-ferret and anti-rabbit IgG 1:200-1:2000. Antibody concentrations can be experimentally selected for any FITC-conjugated antibodies. Any other fluorescent label can be applied for antibody detection if other types of techniques for IIFT are used. The person skilled in the art is familiar with the methods described in (Aoki et al., 2010; Immunofluorescence Test—an Overview|ScienceDirect Topics, n.d.).

In step 635, the attached antibodies, the anti-dog, anti-cat, anti-ferret, or anti-rabbit IgG antibodies, if any, are conjugated with FITC, which makes them visible by incubation for 30 minutes at room temperature, avoiding direct sunlight. In step 640, BIOCHIP slides are washed with PBS-Tween for at least 5 minutes on a rotary shaker, if available, to remove unbound antibodies from the surface. In step 645, the BIOCHIP slide is dried to remove PBS-Tween. In step 650, the BIOCHIP side was placed down onto the cover glass with the mounting media, ensuring a proper fit into the slide's recesses. In step 655, the test results are read and recorded with any suitable fluorescence microscope. A person skilled in art is familiar with the methods described in (Aoki et al., 2010; Huang et al., 2012). Recommended microscope objectives are 20× or 40×, excitation filter of 450-490 nm, a color separator at 510 nm, and a blocking filter at 515 nm.

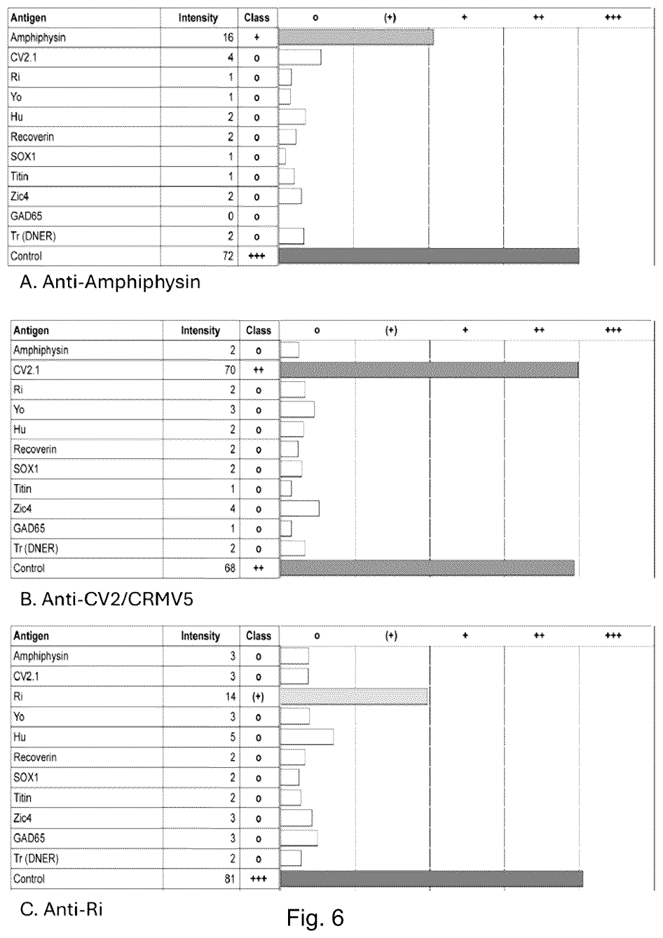

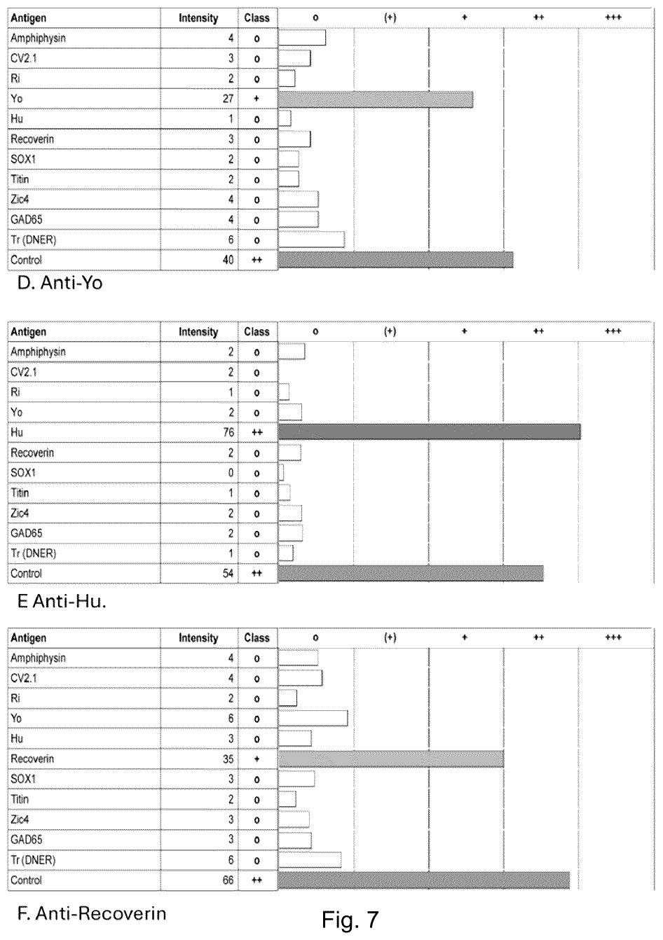

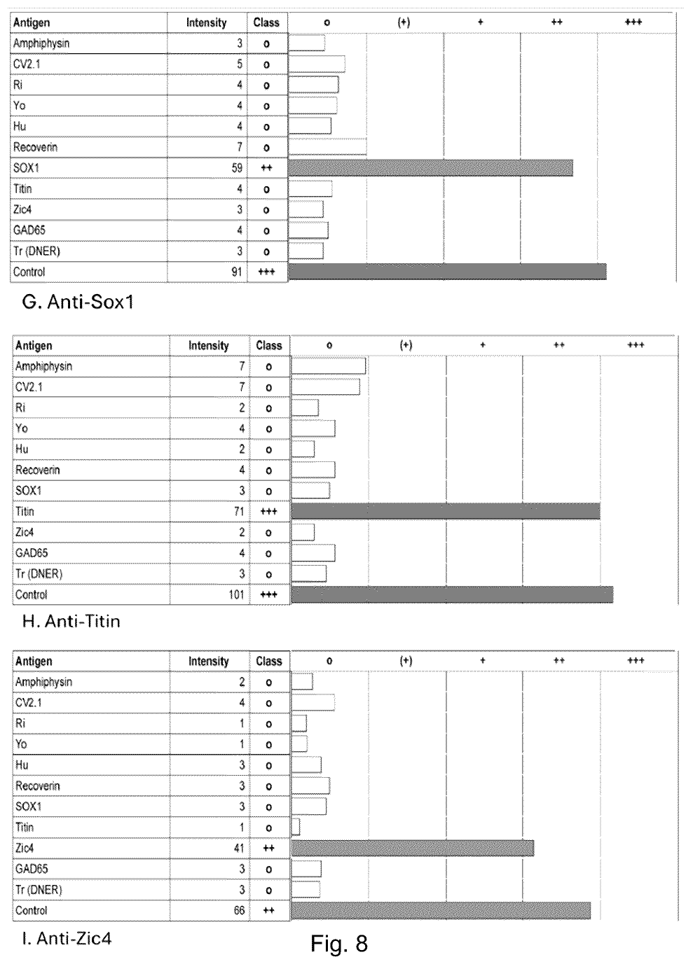

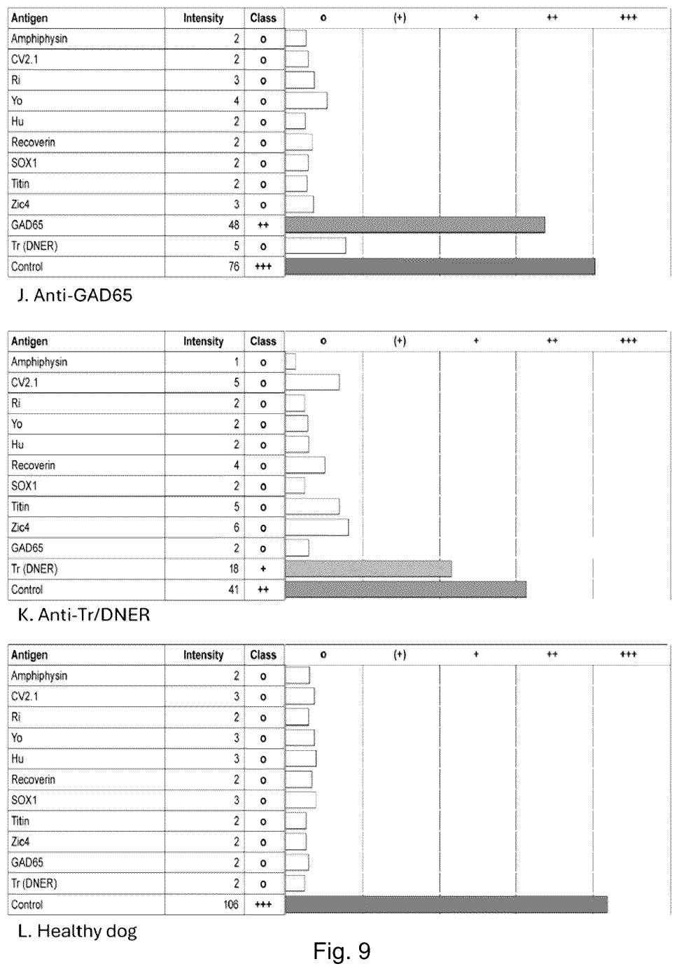

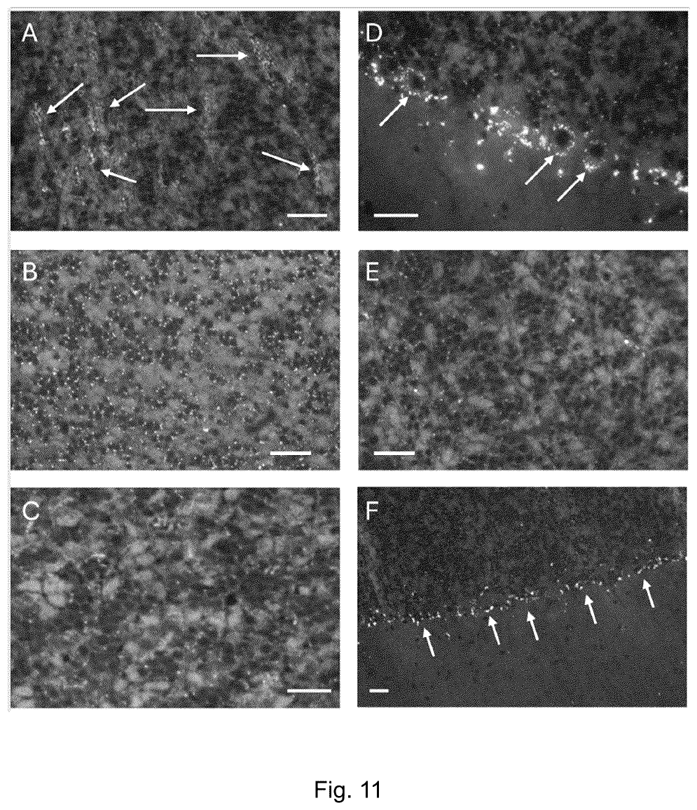

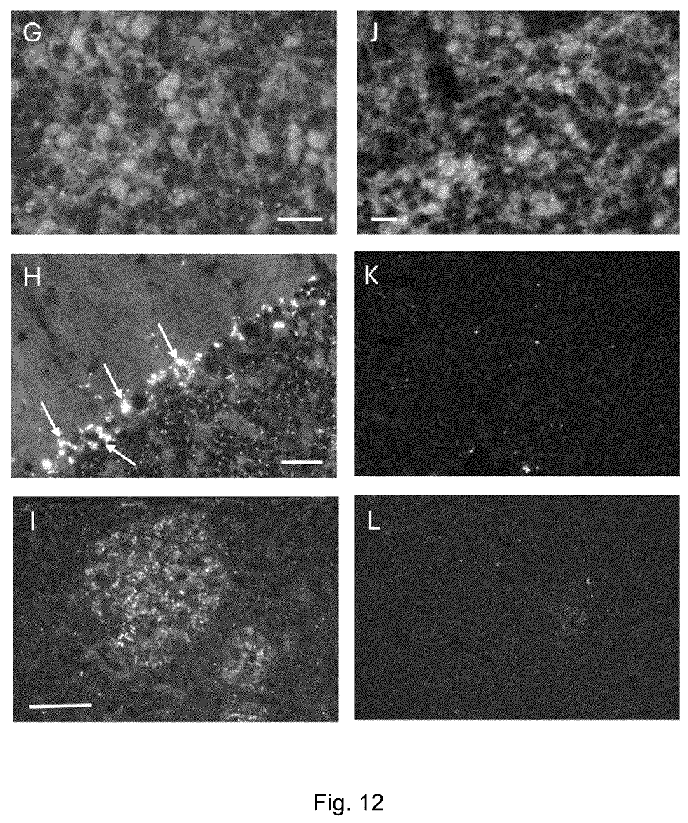

Examples described below are discussed with some reference to FIGS. 11 and 12, which illustrate immunofluorescent detection of ONHR antibodies in the same serums used for the results represented in FIGS. 6-9 and collected from cancer-diagnosed (panels A-J) and cancer-free dogs (panels K, L).

Serums from cancer-diagnosed dogs demonstrate positive staining for onconeural antibodies on the cerebellum tissue sections (panels A-H, J): A—reaction product observed in the presynaptic nerve ends (arrows) of the granular layer (anti-amphiphysin); B—sand-like staining in granular layer (CV2); C—reaction product on almost all neuronal nuclei of the granular layer (anti-Ri); D—arrows point to the strong positive staining of Purkinje cell cytoplasm (anti-Yo); E—reaction product in almost all neuronal nuclei of the granular layer (anti-Hu); F—arrows point to the staining in glial cell within the Purkinje cell layer along the border between molecular and granular layers (anti-Sox1); G—staining in almost all granular layer nuclei (anti-Zic4); H—strong reaction product in the Purkinje cell cytoplasm (arrows) (anti-Tr/DNER). I—islet cells on pancreatic tissue with the reaction product (anti-GAD65). J—reaction product in nerve cell nuclei (anti-PNMa2/Ta). No staining was observed in the serum of cancer-free (healthy) dogs on the cerebellum (K) and pancreatic (L) sections (corresponds to FIG. 1 L. Scale bars: 10 mm for A-H; 40 mm for I.

Other onconeural antibodies, such as anti-recoverin and anti-titin antibodies detected by immunoblot in the serum of dogs diagnosed with cancers have not been supported by IIFT study of monkey cerebellum tissue because they are the antibodies targeting cells in retina and striated muscles. However, these two types of detected antibodies in the serum of dogs diagnosed with specific cancers are correlated with clinical evaluation and symptoms associated with the diagnosed cancer types.

### Example 1—Confirmation of the Presence of ONHR Antibodies to Selected Intracellular Neuronal Antigens in a Canine Subject

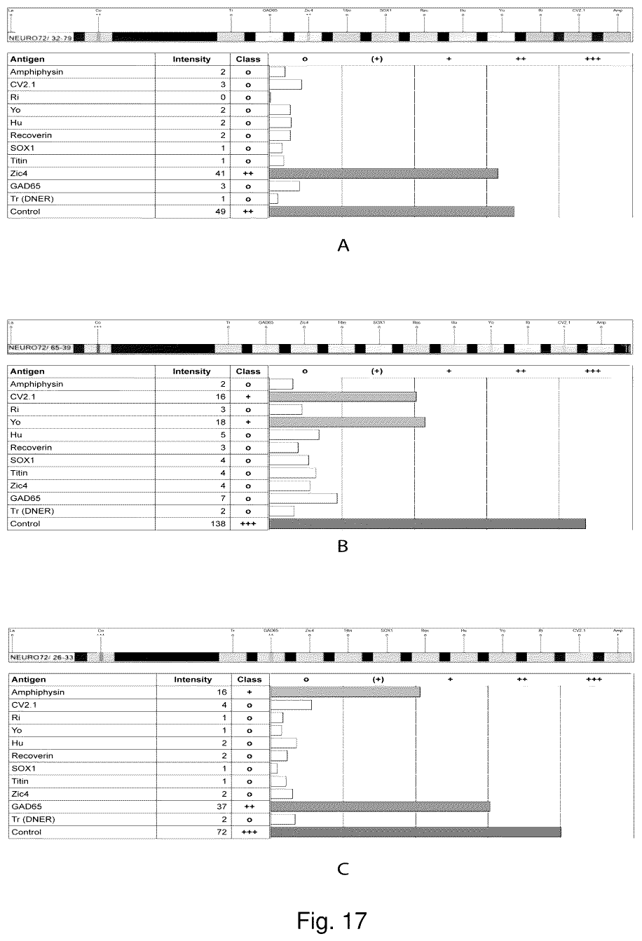

Detection of a single specific ONHR antibody in canine patients diagnosed with several types of cancer is now described. A serum sample from a Wire Fox terrier, a 10-year-old male dog, neutered, previously diagnosed with lymphoma, was sent from a veterinary clinic to determine if it contains any ONHR antibodies. First, the immunoblot analysis revealed the band related to anti-Zic4 antibodies in this patient sample (FIG. 17, panel A). The results obtained by immunoblot have been confirmed by IIFT staining. The anti-Zic4 antibodies against intracellular target antigen zinc finger proteins were detected in the neuronal nuclei of the granular layer of the cerebellum, where this protein is localized (FIG. 12, panel G).

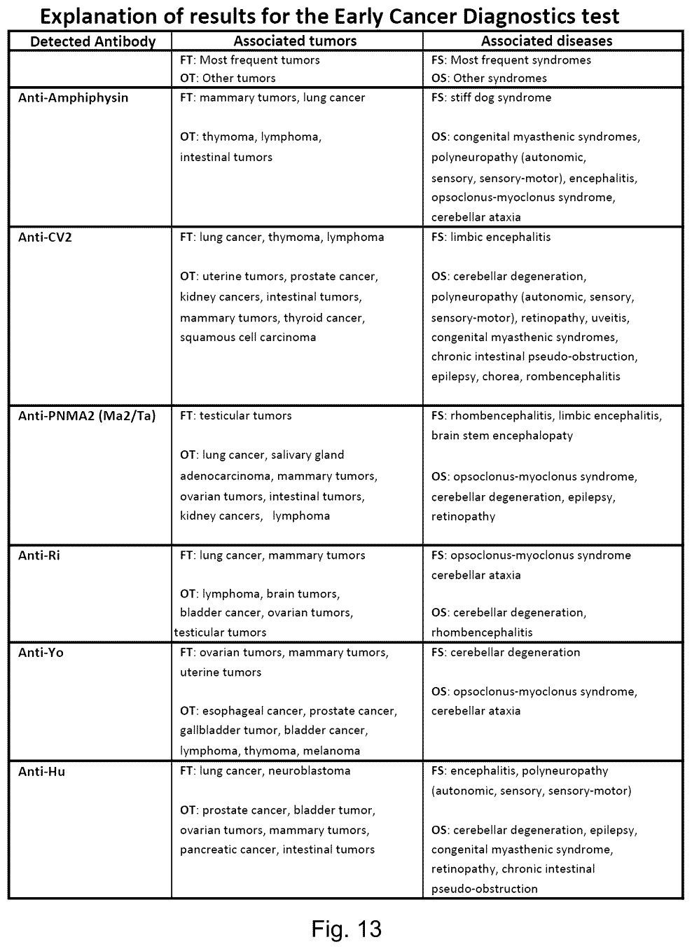

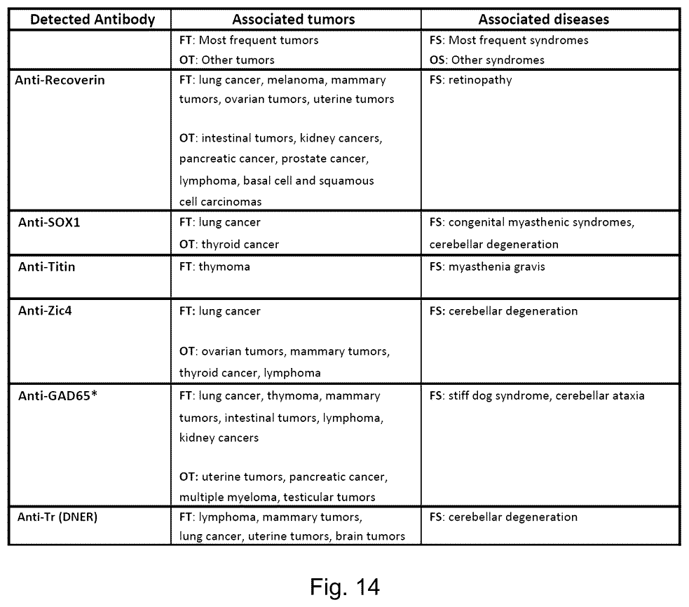

Thus, two different analyses have supported the presence of anti-ZIC4 antibodies in the subject's serum. Results were evaluated by two independent observers. According to the explanation of the results (FIGS. 13 and 14), the presence of anti-Zic4 antibodies in the serum of this subject, confirmed by two different assays, can be associated with lymphoma. The relation of anti-Zic4 antibodies with lymphoma has been published in the Journal of Neuroscience (Eye et al., 2018). The diagnosis of lymphoma in this patient has been supported by two different analyses and demonstrates the unexpected and yet successful application of these diagnostic assays (which were developed for and are used for human diagnostics in a different field of medicine) to canine cancer diagnostics.

### Example 2—Detection of Multiple Specific ONHR Antibodies in Canine Subjects with Cancer

A serum sample from a mixed-breed 12-year-old male dog, neutered, previously diagnosed with prostate carcinoma, has been sent from a veterinary clinic to determine the presence of ONHR antibodies. Immunoblot analysis detected a high level of anti-CV2 and anti-Yo antibodies (FIG. 17, panel B). IIFT confirmed the results of the immunoblot assay by revealing the anti-CV2 (FIG. 11, panel B) and anti-Yo antibodies (FIG. 11, panel D).

The CV2 antibody is now known as a high-risk antibody, and its presence indicates a potential tumor (Graus et al., 2021). It has been published that about 90% of CV2/CRMP5 antibodies are associated with tumors (Budhram et al., 2018; Yu et al., 2001). The detection of these ONHR antibodies can accompany prostate cancer according to the publications (Aliprandi et al., 2015) and to the explanation of the results (FIGS. 13 and 14). The anti-Yo antibodies are also highly associated with some types of cancer, including prostatic adenocarcinoma (Matschke et al., 2007). The presence of these two ONABs has been confirmed by IIFT and demonstrated a positive reaction with the molecular layer and Purkinje cells of the cerebellum. The diagnosis of prostate cancer in this canine subject had also been confirmed by medical examination, blood work, and imaging analysis. Thus, two different tests detected the presence of ONHR antibodies in the subject's serum, confirming the diagnosis of prostate carcinoma.

### Example 3—Detection of ONHR Antibodies in a Canine Suspected of Having Cancer

A mix-breed fourteen-year-old female dog, spayed, had the symptoms of lethargy, muscle weakness, and some instances of discoordination. However, the OncoK9 test did not detect cancer-associated genomic alterations in the DNA from this subject's blood, which means that no cancer was detected. Nevertheless, according to the subject's symptoms that became even worse, the veterinarian ordered the serological test for this subject for ONHR antibody detection. The immunoblot analysis detected a high level of anti-amphiphysin and anti-GAD65 antibodies in the serum sample of this subject (FIG. 17, panel C). The IIFT confirmed the presence of anti-amphiphysin antibodies in the granular and molecular layers of the cerebellum (FIG. 11, panel A), and anti-GAD65 antibodies in the primate pancreas tissue (FIG. 12, panel 1). Due to the similar localization of GAD65 and amphiphysin antigens in the granular layer of cerebellum, the pancreas tissue has been used for anti-GAD65 antibody representation (FIG. 12, panel 1).

In humans, anti-GAD65 antibodies are the most common autoantigen in subjects with different carcinomas associated with stiff person syndrome spectrum disorders (SPSSDs) (Peng et al., 2023) (Ariño et al., 2014; Peng et al., 2023). The anti-amphiphysin antibodies are also strongly associated with these two paraneoplastic disorders (Murinson & Guarnaccia, 2008). Amphiphysin antibodies are associated with various paraneoplastic neurological syndromes and tumors (Antoine et al., 1999). Although neurological syndromes are rare in animals, the subject's symptoms were similar to symptoms of cerebellar ataxia, which led to a search for underlying tumors. According to the onconeural antibody test results and the symptoms, the follow-up analyses, including X-ray, blood count, chemistry profile, and others, revealed lesions in the pelvis and proximal femur, which are common locations in myeloma. Thus, based on onconeural antibodies tests and other assays, this subject was diagnosed with multiple myeloma and finally euthanized in 9 months after an anti-GAD65 antibody detection.

### Example 4—Cancer Screening According to Breed and Age Predispositions

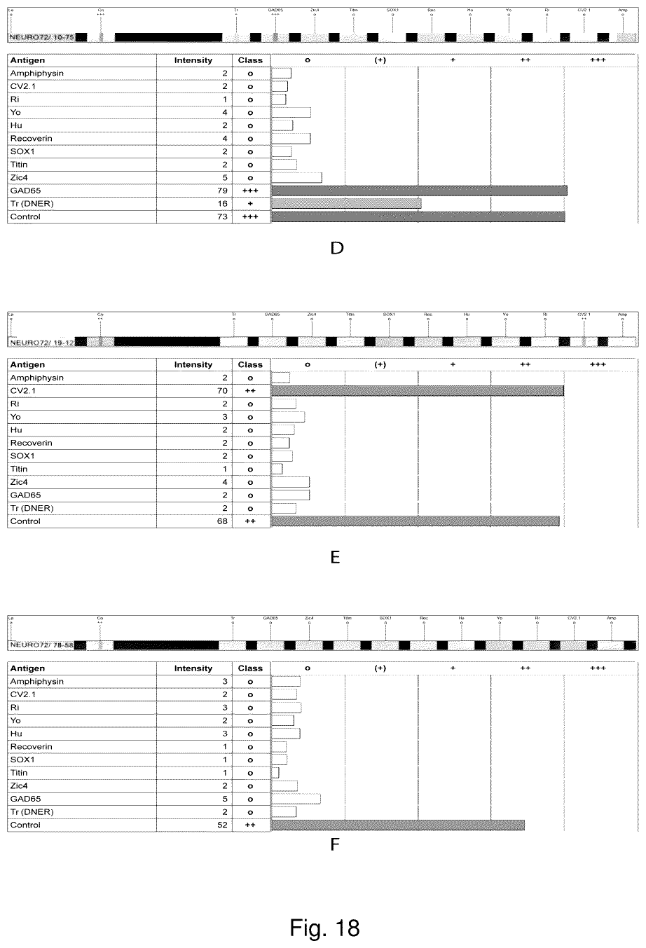

English bulldog, six-year-old, female, spayed. The dog, without any symptoms or signs of the disease in a healthy condition, was analyzed just due to a breed predisposition. The immunoblot analysis revealed a high level of anti-GAD65 and anti-Tr (DNER) antibodies (FIG. 18, panel D). The IIFT detected anti-GAD65 antibodies in the primate pancreas tissue (FIG. 12 I) as well as anti-Tr (DNER) antibodies in Purkinje cells of the cerebellum (FIG. 12, panel H).

In nine months after the ONHR test, gastrointestinal ulcers have been found. The dog is still in good condition but needs evaluation every two months because lymphoma is suspected. Thus, the test for ONHR antibody detection can be used as a preventative test for the detection of cancer at its early stage.

### Example 5—Monitoring of Cancer Treatment by Onconeural Antibodies Analysis

A boxer, four-year-old female, spayed. The subject had a suspicious nodule on the side that looked like a skin tag that was aspirated for histopathology, and a serum sample was sent to ONHR antibody analysis. Histopathology results have demonstrated the mast cell tumor (MCT). The immunoblot analysis showed the presence of a relatively high level of anti-CV2 antibodies (FIG. 18, panel E). The IIFT has also detected anti-CV2 in the molecular layer of the cerebellum (image like FIG. 11, panel B).

After the ONHR and IIFT analysis, a low-grade tumor was completely excised. Four months later, serum from this subject was analyzed for the presence of ONHR antibodies again. No ONHR antibodies were detected in the subject's serum four months after the tumor removal (FIG. 18, panel F). Thus, this test can be used to monitor treatment.

### Example 6—Healthy Control

Great Pyrenees, eight-year-old male, neutered. The dog without any symptoms or signs of cancer in a healthy condition has been used as a negative control. This subject's serum has been used for analysis of the absence of ONHR antibodies. The immunoblot did not detect any ONHR antibodies in the serum of the subject (image like FIG. 18, panel F). The IIFT confirmed the results of the immunoblot. No reaction product was observed on cerebellar and pancreatic tissues. (images like FIG. 12, panels K, L).

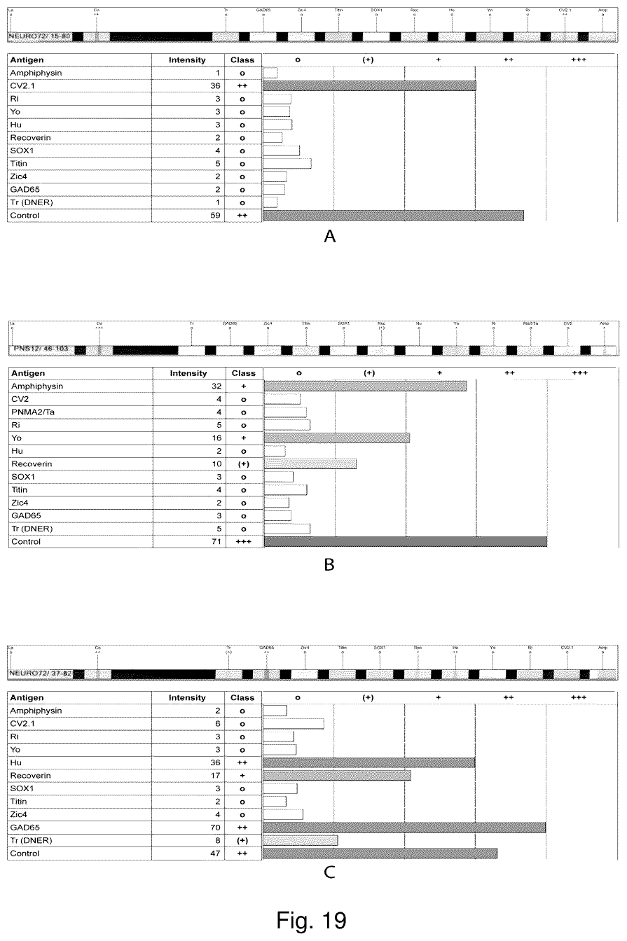

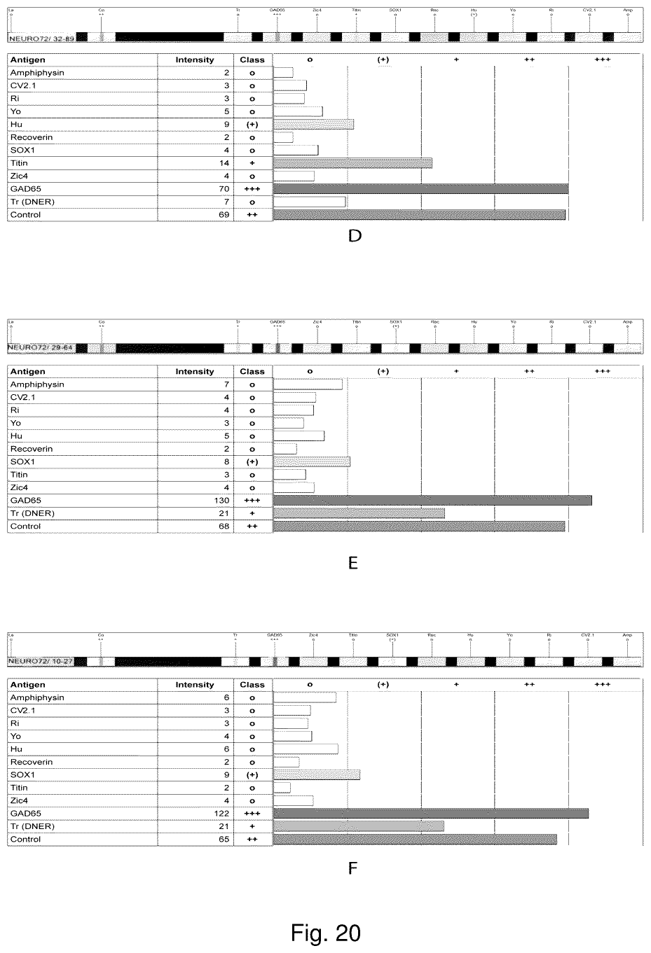

### Example 7—Confirmation of the Presence of ONHR Antibodies to Selected Intracellular Neuronal Antigens in Feline Subject Samples (FIGS. 19 and 20, Panels A-F)

Single ONHR antibody detected in a feline subject diagnosed with associated types of cancer is now described. A 10-year-old DSH, male, neutered, is diagnosed with lymphoma. The immunoblot analysis detected the anti-CV2 antibodies (FIG. 19, panel A). The results of the immunoblot assay have been confirmed by IIFT, which showed the positivity to anti-CV2 antibodies in the molecular layer of the cerebellum (image like FIG. 11, panel B). According to the explanation of the results (FIGS. 13 and 14), the presence of these antibodies can be associated with lymphoma. Thus, ONHR antibody detection is a valuable test for feline cancer diagnostics.

### Example 8—Multiple Onconeural Antibodies

A 9-years-old Main Coon female, spayed, diagnosed with mammary gland carcinoma (MGC) having grade 3 pulmonary metastasis. The immunoblot analysis revealed a high concentration of anti-amphiphysin and anti-Yo antibodies (FIG. 19, panel B). The IIFT confirmed the results of immunoblot analysis and also detected anti-amphiphysin antibodies localized in nerve ends of cerebellum (image like FIG. 11, panel A) and anti-Yo antibodies in cytoplasm of Purkinje cells (image like FIG. 11, panel D).

Mammary cancer is the third most common feline cancer. Based on a full physical exam that was focused on mass, which could be palpated, the X-ray and abdominal ultrasounds have shown that the tumor has spread to the lung. Cats with advanced lung involvement at the time the tumor is diagnosed have a median survival time of only one month. Histopathological analysis of the biopsy has revealed a grade 3 carcinoma.

Besides the procedures described above, basic blood tests including a complete blood count, serum chemistry panel, and ONHR antibody detection have been performed. The serum sample from this subject was analyzed according to the explanation of the results (FIGS. 13 and 14). The presence of anti-amphiphysin and anti-Yo antibodies can be associated with mammary gland carcinoma. Thus, the ONHR antibody detection is a valuable test for feline cancer diagnostics.

### Example 9—Detection of ONHR Antibodies in Felines with Suspicious Cancer

The subject is a mix-breed 7-year-old neutered male with suspected cancer due to some symptoms, including a lack of energy, decreased physical activities, loss an appetite, and some weight loss, with a periodical incident of difficulties in breathing. Based on a complete physical examination and according to symptoms, the serum sample from this subject was sent for early cancer diagnosis that assays for the ONHR antibodies presence in blood. The immunoblot analysis confirmed the presence of high levels of anti-Hu, anti-GAD65, anti-recoverin antibodies, and a very low level of anti-Tr (DNER) antibodies (FIG. 19, panel C). The IIFT showed the positivity for anti-Hu antibodies in neuronal nuclei on the cerebellum substrate (image like FIG. 11, panel E) and anti-GAD65 antibody localized in islet cells in the pancreas, which confirmed the results of the immunoblot assay (image like FIG. 12, panel 1).

Blood work and a biochemistry profile, as well as an X-ray of internal organs, were recommended to evaluate the subject's physical condition. Hematology and biochemistry have demonstrated eosinopenia (0.04×109/l; RI 0.1-1.49×109/l), anemia, hypokalemia (2.9 mmol/l; RI 3.5-5.8), and hyperglycemia (10 mmol/l; RI 4-8 mmol/l), which can be associated with stress or disease. No pulmonary lesions were evident by thoracic radiographs. Five weeks after the first visit, the subject was reviewed, and an X-ray was repeated. The second plain radiograph showed two pulmonary masses in the right lung lobe. The most likely diagnosis based on blood work, biochemistry, X-ray, and ONHR antibody assays, according to the explanation of the results (FIGS. 13 and 14), was primary pulmonary adenocarcinoma. Thus, the detection of ONHR antibodies can be used as an early cancer diagnostic test.

### Example 10—Breed and Age Predisposition to Cancer in Cats

A sample from a 12-year-old Siamese neutered male was tested because of a weight loss, overall weakness and continuous coughing. The immunoblot analysis revealed the presence of anti-GAD65 and anti-Titin antibodies at a high level, and also a low level of anti-Hu antibodies (FIG. 20, panel D). The IIFT has detected the anti-GAD65 antibodies localized in islet cells in the pancreas (image like FIG. 12, panel 1).

A physical examination and several other tests have shown eosinophilia and leukocytosis. According to the explanation of the results (FIGS. 13 and 14), the increased level of ONHR antibodies in subject serum can be associated with thymoma. Anti-Hu antibody has been detected only at a low level and is not included in the explanation of the results in association with thymoma. Some case reports revealed the detection of this antibody in subjects diagnosed with thymoma (Ricigliano et al., 2018)

Thymus tumors are uncommon in cats, and diseases are usually detected in older cats. Main Coons are also predisposed to thymoma. Based on the ONHR antibody test results, a chest radiograph was performed that showed a mass between the lungs. Thus, the ONHR antibody test can be recommended for early cancer detection in cats.

### Example 11—Monitoring a Subject's Health Condition with Onconeural Antibodies Analysis in Cats

The pet owner of a fourteen-year-old DLH neutered male suggested the cat has cancer based on respiratory symptoms. The first ONHR antibody test was performed on May 28, 2024. The immunoblot has detected very high levels of anti-GAD65 antibodies, a high level of anti-Tr (DNER) antibodies, and a borderline low level of antibodies against transcription factor SOX1 (FIG. 20, panel E). The IIFT confirmed the presence of anti-Tr (DNER) antibodies localized in the granular pattern of the cytoplasm of Purkinje cells in the cerebellum tissue (image like on FIG. 12, panel H).

No cancer treatment was initiated. In five months, the respiratory symptoms in this subject became worse. The second test was performed on the cat subject on Oct. 9, 2024. The immunoblot again detected very high levels of anti-GAD65, a high level of anti-Tr (DNER) and a very low level of SOX1 antibodies (FIG. 20, panel F). The IIFT confirmed the results of immunoblot and revealed the reaction product of anti-Tr (DNER) in the granular pattern in the cytoplasm of Purkinje cells in the cerebellum tissue (image like FIG. 12, panel H) and for anti-GAD65 in pancreatic tissue (image like FIG. 12, panel 1). Chest X-ray and bloodwork did not find any evidence of the disease. However, it has been published that in 90% of cases, errors in diagnosis leading to missed lung cancer occur on chest radiographs because of tumor characteristics, technical considerations, or observer error (del Ciello et al., 2017). The onconeural antibodies test results were highly abnormal. It is known that onconeural antibodies can be developed months or even years before tumor formation. Thus, this subject needs to be evaluated every two or three months to avoid misdiagnoses and get appropriate treatment in time. In five months, the respiratory symptoms in this subject became worse. According to these results, the onconeural antibodies test can be used to monitor a subject's condition or treatment if initiated.

### Example 12—Feline Healthy Control

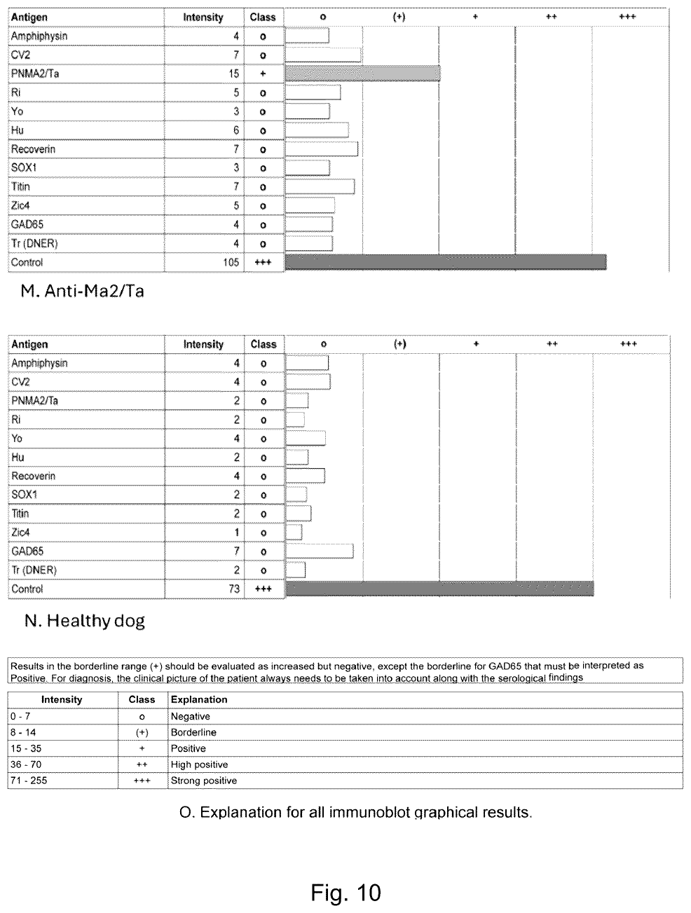

The subject is a fourteen-year-old spayed female DSH without any symptoms or signs of the disease in a healthy condition. This subject's serum has been used for analysis for ONHR antibody presence. The immunoblot (image like FIG. 10, panel N (of note is that this image is that for a dog, but a cat image is identical and therefore omitted)) and IIFT (image like FIG. 12, panels K, L) analysis did not reveal any ONHR antibodies in the cat's serum sample.

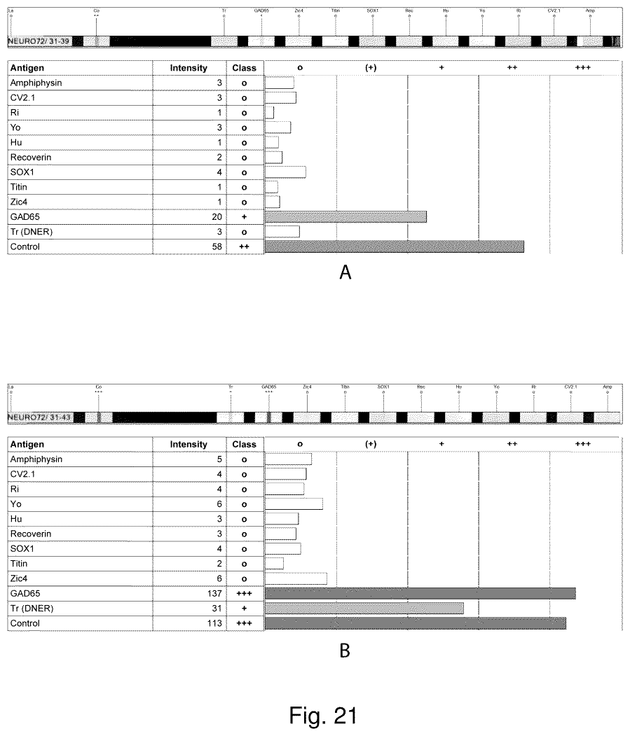

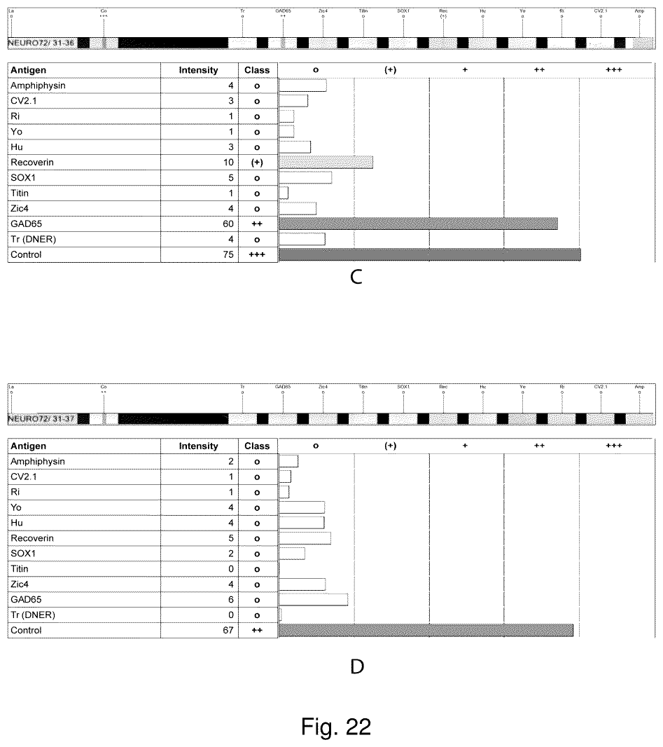

Confirmation of the presence of ONHR antibodies to selected intracellular neuronal antigens in musteline subject samples is now described and illustrated in FIGS. 21 and 22, panels A-D.

### Example 13—Detection of Specific ONHR Antibodies in a Ferret Subject Diagnosed with Cancer

A four-year-old spayed female Marshalls subject was diagnosed with thymoma, supported by histopathological analysis. A serum sample was also sent for ONHR antibody detection. The immunoblot detected a very high level of anti-GAD65 antibodies (FIG. 21, panel A). These results have been supported by IIFT (image like FIG. 12, panel I). According to the explanation of the results (FIGS. 13 and 14), detection of antibodies against glutamic acid decarboxylase GAD65 in the serum, confirmed by two different methods, can be associated with underlying thymoma.

### Example 14—Detection of ONHR Antibodies in Ferrets with Suspicious Cancer

A 7-year-old neutered Parks ferret tested positive for anti-GAD65 and anti-Tr (DNER) antibodies (FIG. 21, panel B), which confirmed the veterinarian's suspicion of lymphoma. The IIFT assay of serum confirmed the immunoblot's results for anti-Tr (DNER) antibodies (image like FIG. 12, panels H) and for anti-GAD65 antibodies (image like FIG. 12, panel 1). The explanation of the results suggested the lymphoma as a diagnosis (FIGS. 13 and 14).

### Example 15—Detection of ONHR Antibodies in a Presumably Cancer-Free Ferret

A six-year-old spayed Marshall's ferret, without any symptoms of the disease, was used as a healthy control for ONHR antibody detection. Interestingly, the immunoblot detected a high level of the anti-GAD65 antibodies and a low level (borderline) of anti-recoverin antibodies (FIG. 22, panel C). The IIFT assay has confirmed the presence of anti-GAD65 antibodies (image like FIG. 12, panel I).

A month after getting the ONHR antibody test results, some symptoms were detected, such as mild weakness. High insulin level and low blood sugar indicated pancreatic problems. According to the explanation of the results (FIG. 14), the identification of anti-GAD65 and anti-recoverin antibodies has been related to pancreatic cancer (insulinoma), which is a common and serious cancer in ferret subjects. Thus, ONHR antibody detection is a helpful method for early cancer diagnosis in ferrets and can be used as a screening test to prevent the development of cancers via effective treatment.

### Example 16—Detection of “High-Risk” Antibodies in Cancer-Free Ferrets

A 2-year-old Marshalls neutered ferret without any symptoms or signs of the disease was used to confirm its healthy condition and as a healthy control. This subject's serum has been analyzed for ONHR antibody presence. Neither immunoblot (FIG. 22, panel D) nor IIFT (image like FIG. 12, panels K, L) indicated a cancer.

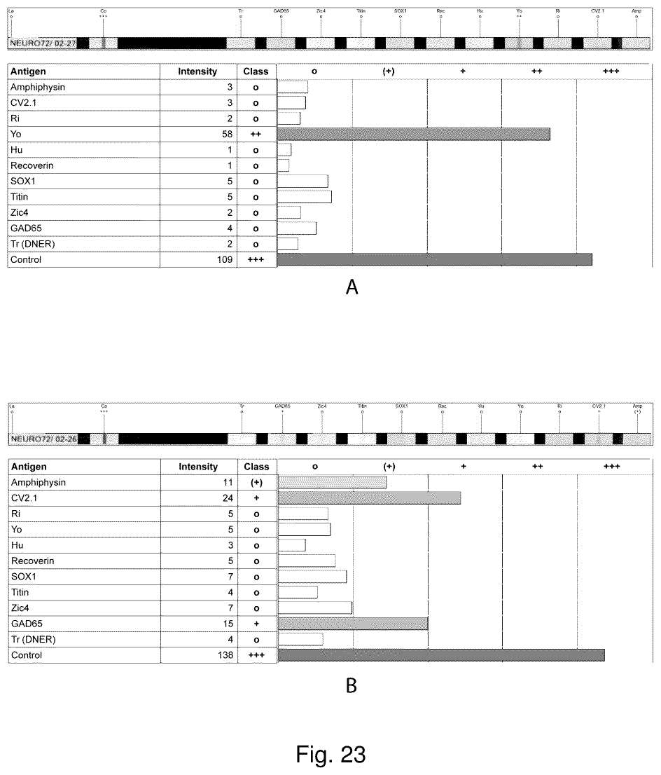

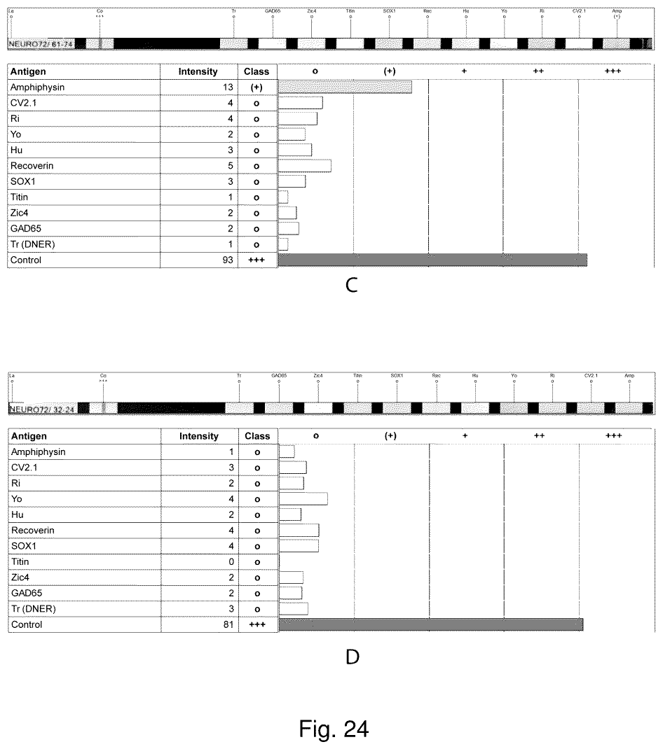

Confirmation of the presence of ONHR antibodies to selected intracellular neuronal antigens in leporine subject samples is now described in greater detail and illustrated in FIGS. 23 and 24, panels A-D.

### Example 17—Detection of ONHR Antibodies in a Rabbit Diagnosed with Cancer

A standard eight-year-old female rabbit has been diagnosed with a uterine tumor. The immunoblot detected the anti-Yo antibodies (FIG. 23, panel A). The IIFT confirmed the presence of anti-Yo antibodies (image like FIG. 11, panel D). Two different methods confirmed the presence of anti-Yo ONHR antibodies, which, according to the Explanation of the results (FIG. 14), can be associated with uterine tumor, the most common cancer affecting unspayed rabbits. The method of the invention may, therefore, be used for the diagnosis of cancer in rabbit subjects.

### Example 18—Detection of ONHR Antibodies in Rabbits Suspected of Having Cancer

The subject is a 4-year-old Holland Lop spayed rabbit, low in activity, which may be a mild symptom of a possible disease. The immunoblot revealed a high level of anti-CV2 and anti-GAD65 antibodies and a low level (borderline) of anti-amphiphysin antibodies (FIG. 23, panel B). The IIFT confirmed the presence of anti-CV2 antibodies at the molecular level of the cerebellum (image like FIG. 11, panel B), and anti-GAD65 antibodies localized in islet cells in the pancreas (image like FIG. 12, panel 1).

According to the explanation of the results (FIGS. 13 and 14), the subject's condition can be associated with underlying lymphoma, a relatively common cancer in rabbit subjects. Three months after the test, skin nodules showed up. Biopsy, histopathological, and immunocytochemical analyses have supported the diagnosis of lymphoma. Thus, the method of the invention can be used for early cancer identification in rabbits.

### Example 19—Detection of ONHR Antibodies in a Presumably Cancer-Free Rabbit

A 7-year-old Lion's head spayed rabbit subject without any symptoms of the disease was tested and intended to be used as a healthy control. The immunoblot analysis identified a relatively low level of anti-amphiphysin antibodies (FIG. 24, panel C). The IIFT assay has detected the antibodies against synaptic protein amphiphysin localized in the molecular and granular layer of the cerebellum (image like FIG. 11, panel A). Due to some abnormalities in test results, the subject has been scheduled for follow-up in two months. At that time, this rabbit started to demonstrate some mild breathing difficulties. Physical examination and chest radiography confirm the thymoma. According to the explanation of the results and the onconeural antibody detection, the increased level of anti-amphiphysin antibodies can be associated with thymoma. The subject is still alive and has been treated with radiation therapy. Thus, the method of the invention can be used for early cancer detection in rabbits and instituting a timely cancer therapy.

### Example 20—Detection of ONHR Antibodies in a Cancer-Free Rabbit

A two-year-old Wild and Domestic mix neutered rabbit subject without any symptoms or signs of a disease in a healthy condition has been used as a healthy control. Neither immunoblot (FIG. 24, panel D) nor IIFT (image like FIG. 12, panels K, L) indicated a cancer.

Overall, the present invention provides a method and examples of a qualitative immunoblot-based in vitro method for detecting onconeural antibodies class IgG to a number of different antigens in serum or plasma of a mammalian animal, which can be used for early diagnosis of cancer and cancer-associated neurological diseases.

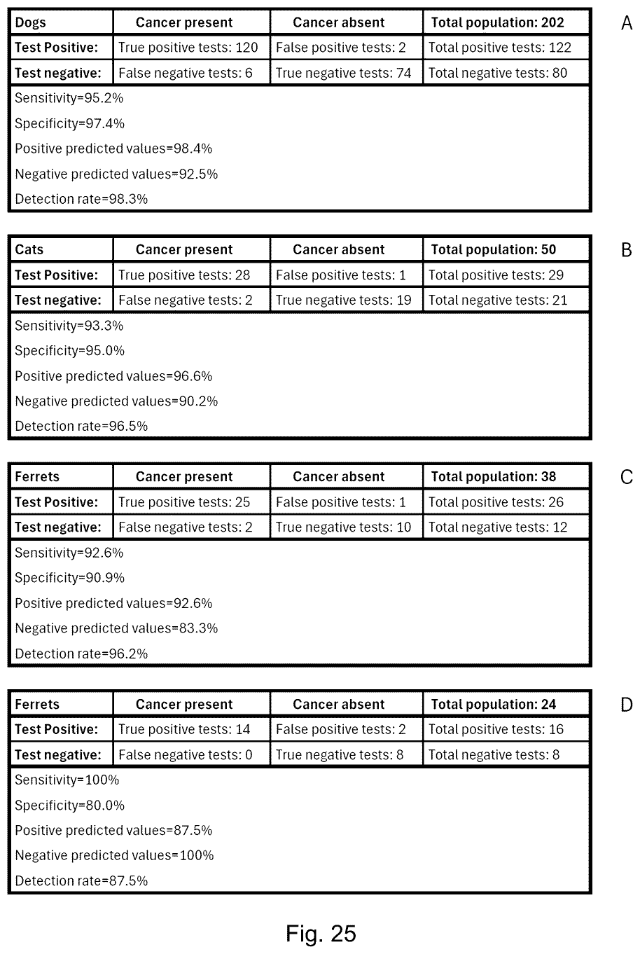

The recognition of onconeural antibodies in the blood of the abovementioned animals is supported by an indirect immunofluorescent assay. The immunoblot-based method has been used for the detection of paraneoplastic neurological syndrome. The method is adopted for the early detection of cancer in animals due to the uncommon neurological diseases associated with malignancy in animals. Enzyme anti-dog, anti-cat, anti-ferret, and anti-rabbit conjugates and serum or plasma quantity have been experimentally selected and approved for each animal species. Overall, the Early Cancer Diagnostics method demonstrated high sensitivity, specificity, and ability to serve as a new, groundbreaking Early Cancer Diagnostics test in Veterinary medicine for at least the following four species: dogs, cats, ferrets, and rabbits (see FIG. 25).

### REFERENCES

1. Aliprandi, A., Terruzzi, A., Rigamonti, A., Bazzigaluppi, E., Tremolizzo, L., Ferrarese, C., & Salmaggi, A. (2015). Paraneoplastic cerebellar degeneration with anti-CV2/CRMP5 antibodies and prostate adenocarcinoma. In Neurological Sciences (Vol. 36, Issue 8). https://doi.org/10.1007/s10072-015-2113-5
2. Antoine, J. C., Absi, L., Honnorat, J., Boidesteix, J. M., De Brouker, T., Vial, C., Butler, M., Camilli, P. D. C., & Michel, D. (1999). Antiamphiphysin antibodies are associated with various paraneoplastic neurological syndromes and tumors. Archives of Neurology, 56(2). https://doi.org/10.1001/archneur.56.2.172
3. Aoki, V., Fukumori, L. M. I., Freitas, E. L., Sousa, J. X., Périgo, A. M., & Oliveira, Z. N. P. (2010). Direct and indirect immunofluorescence. In Anais Brasileiros de Dermatologia (Vol. 85, Issue 4). https://doi.org/10.1590/s0365-05962010000400010
4. Ariño, H., Gresa-Arribas, N., Blanco, Y., Martínez-Hernández, E., Sabater, L., Petit-Pedrol, M., Rouco, I., Bataller, L., Dalmau, J. O., Saiz, A., & Graus, F. (2014). Cerebellar Ataxia and Glutamic Acid Decarboxylase Antibodies. JAMA Neurology, 71(8). https://doi.org/10.1001/jamaneurol.2014.1011
5. Arunprasath, P., Rai, R., & Venkataswamy, C. (2020). Comparative Analysis of BIOCHIP Mosaic-Based Indirect Immunofluorescence with Direct Immunofluorescence in Diagnosis of Autoimmune Bullous Diseases: A Cross-Sectional Study. Indian Dermatology Online Journal, 11(6). https://doi.org/10.4103/idoj.IDOJ_156_20
6. Binks, S., Lamquet, S., Crawford, A. H., Meurs, A., Irani, S. R., & Pakozdy, A. (2022). Parallel roles of neuroinflammation in feline and human epilepsies. Veterinary Journal, 290. https://doi.org/10.1016/j.tvjl.2022.105912
7. Budhram, A., Nicolle, M. W., & Yang, L. (2018). The Positive Predictive Value of Onconeural Antibody Testing: A Retrospective Review. In Canadian Journal of Neurological Sciences (Vol. 45, Issue 5). https://doi.org/10.1017/cjn.2018.74
8. Budhram, A., & Sechi, E. (2024). Antibodies to neural cell surface and synaptic proteins in paraneoplastic neurologic syndromes. In Handbook of Clinical Neurology (Vol. 200). https://doi.org/10.1016/B978-0-12-823912-4.00006-2
9. Chatterjee, M., Hurley, L. C., & Tainsky, M. A. (2017). Paraneoplastic antigens as biomarkers for early diagnosis of ovarian cancer. In Gynecologic Oncology Reports (Vol. 21). https://doi.org/10.1016/j.gore.2017.06.006
10. Chen, W., Tang, D., Tang, D., & Dai, Y. (2020). Epigenetic silencing of ZIC4 contributes to cancer progression in hepatocellular carcinoma. Cell Death and Disease, 11(10). https://doi.org/10.1038/s41419-020-03109-1
11. D'Alessandro, V., Muscarella, L. A., la Torre, A., Bisceglia, M., Parrella, P., Scaramuzzi, G., Storlazzi, C. T., Trombetta, D., Kok, K., De Cata, A., Sperandeo, M., Zelante, L., Carella, M., & Vendemiale, G. (2010). Molecular analysis of the HuD gene in neuroendocrine lung cancers. Lung Cancer, 67(1). https://doi.org/10.1016/j.lungcan.2009.03.022
12. Darnell, J. C., Albert, M. L., & Darnell, R. B. (2000). cdr2, A target antigen of naturally occurring human tumor immunity, is widely expressed in gynecological tumors. Cancer Research, 60(8).
13. Davison, L. J., Weenink, S. M., Christie, M. R., Herrtage, M. E., & Catchpole, B. (2008). Autoantibodies to GAD65 and IA-2 in canine diabetes mellitus. Veterinary Immunology and Immunopathology, 126(1-2). https://doi.org/10.1016/j.vetimm.2008.06.016
14. Déchelotte, B., Muñiz-Castrillo, S., Joubert, B., Vogrig, A., Picard, G., Rogemond, V., Pinto, A. L., Lombard, C., Desestret, V., Fabien, N., & Honnorat, J. (2020). Diagnostic yield of commercial immunodots to diagnose paraneoplastic neurologic syndromes. Neurology: Neuroimmunology and NeuroInflammation, 7(3). https://doi.org/10.1212/NXI.0000000000000701
15. del Ciello, A., Franchi, P., Contegiacomo, A., Cicchetti, G., Bonomo, L., & Larici, A. R. (2017). Missed lung cancer: When, where, and why? In Diagnostic and Interventional Radiology (Vol. 23, Issue 2). https://doi.org/10.5152/dir.2016.16187
16. Dolan, C., Miller, T., Jill, J., Terrell, J., Kelly, T. K., Bygott, T., & Wilson-Robles, H. (2021). Characterizing circulating nucleosomes in the plasma of dogs with lymphoma. BMC Veterinary Research, 17(1). https://doi.org/10.1186/s12917-021-02991-x
17. Doyle, H. A., & Mamula, M. J. (2012). Autoantigenesis: The evolution of protein modifications in autoimmune disease. In Current Opinion in Immunology (Vol. 24, Issue 1). https://doi.org/10.1016/j.coi.2011.12.003
18. Eye, P. G., Wang, B., Keung, E. S., & Tagg, N. T. (2018). Anti-ZIC4 associated paraneoplastic cerebellar degeneration in a subject with both diffuse large B-cell lymphoma and incidental smoldering multiple myeloma. In Journal of the Neurological Sciences (Vol. 384). https://doi.org/10.1016/j.jns.2017.11.005
19. Fernández-Fournier, M., Lacruz, L., Nozal, P., Chico, J. L., Tallón Barranco, A., Otero-Ortega, L., Corral, I., & Carrasco, A. (2022). The study of neural antibodies in neurology: A practical summary. In Frontiers in Immunology (Vol. 13). https://doi.org/10.3389/fimmu.2022.1043723
20. Flory, A., Kruglyak, K. M., Tynan, J. A., McLennan, L. M., Rafalko, J. M., Fiaux, P. C., Hernandez, G. E., Marass, F., Nakashe, P., Ruiz-Perez, C. A., Fath, D. M., Jennings, T., Motalli-Pepio, R., Wotrang, K., McCleary-Wheeler, A. L., Lana, S., Phillips, B., Flesner, B. K., Leibman, N. F., . . . Tsui, D. W. Y. (2022). Clinical validation of a next-generation sequencing-based multi-cancer early detection “liquid biopsy” blood test in over 1,000 dogs using an independent testing set: The CANcer Detection in Dogs (CANDiD) study. PLoS ONE, 17(4 April). https://doi.org/10.1371/journal.pone.0266623
21. Floyd, S., Butler, M. H., Cremona, O., David, C., Freyberg, Z., Zhang, X., Solimena, M., Tokunaga, A., Ishizu, H., Tsutsui, K., & De Camilli, P. (1998). Expression of amphiphysin I, an autoantigen of paraneoplastic neurological syndromes, in breast cancer. Molecular Medicine, 4(1). https://doi.org/10.1007/bf03401727
22. Glantschnigg-Eisl, U., Klang, A., Kneissl, S., Lang, B., Waters, P., Irani, S. R., Binks, S. N. M., & Pakozdy, A. (2023). A feline model of spontaneously occurring autoimmune limbic encephalitis. Veterinary Journal, 296-297. https://doi.org/10.1016/j.tvjl.2023.105974
23. Glisovic, T., Bachorik, J. L., Yong, J., & Dreyfuss, G. (2008). RNA-binding proteins and post-transcriptional gene regulation. In FEBS Letters (Vol. 582, Issue 14). https://doi.org/10.1016/j.febslet.2008.03.004
24. Godelaine, J., Bossuyt, X., & Poesen, K. (2019). The clinical significance of atypical indirect immunofluorescence patterns on primate cerebellum in paraneoplastic antibody screening. Autoimmunity Highlights, 10(1). https://doi.org/10.1186/s13317-019-0116-6
25. Graus, F., Dalmau, J., Reñé, R., Tora, M., Malats, N., Verschuuren, J. J., Cardenal, F., Viñolas, N., Garcia del Muro, J., Vadell, C., Mason, W. P., Rosell, R., Posner, J. B., & Real, F. X. (1997). Anti-Hu antibodies in subjects with small-cell lung cancer: Association with complete response to therapy and improved survival. Journal of Clinical Oncology, 15(8). https://doi.org/10.1200/JCO.1997.15.8.2866
26. Graus, F., Delattre, J. Y., Antoine, J. C., Dalmau, J., Giometto, B., Grisold, W., Honnorat, J., Smitt, P. S., Yedeler, C., Verschuuren, J. J. G. M., Vincent, A., & Voltz, R. (2004). Recommended diagnostic criteria for paraneoplastic neurological syndromes. In Journal of Neurology, Neurosurgery and Psychiatry (Vol. 75, Issue 8). https://doi.org/10.1136/jnnp.2003.034447
27. Graus, F., Vogrig, A., Muñiz-Castrillo, S., Antoine, J. C. G., Desestret, V., Dubey, D., Giometto, B., Irani, S. R., Joubert, B., Leypoldt, F., McKeon, A., Prüss, H., Psimaras, D., Thomas, L., Titulaer, M. J., Vedeler, C. A., Verschuuren, J. J., Dalmau, J., & Honnorat, J. (2021). Updated Diagnostic Criteria for Paraneoplastic Neurologic Syndromes. Neurology: Neuroimmunology and NeuroInflammation, 8(4). https://doi.org/10.1212/NXI.0000000000001014
28. Haukanes, B. I., Hegvik, T. A., Eichler, T., Haavik, J., & Vedeler, C. (2015). Paraneoplastic syndrome-associated neuronal antibodies in adult ADHD. Journal of Neuroimmunology, 288. https://doi.org/10.1016/j.jneuroim.2015.08.018
29. Hemmeter, L., Bien, C. G., Bien, C. I., Tipold, A., Neßler, J., Bathen-Nöthen, A., Matiasek, K., Dahlhoff, M., Rusbridge, C., Rotter Black, C., Rentmeister, K., Volk, H. A., & Fischer, A. (2023). Investigation of the presence of specific neural antibodies in dogs with epilepsy or dyskinesia using murine and human assays. Journal of Veterinary Internal Medicine, 37(4). https://doi.org/10.1111/jvim.16744
30. Honnorat, J., & Antoine, J. C. (2007). Paraneoplastic neurological syndromes. In Orphanet Journal of Rare Diseases (Vol. 2, Issue 1). https://doi.org/10.1186/1750-1172-2-22
31. Huang, P., Lin, J., Wang, X., Wang, Z., Zhang, C., He, M., Wang, K., Chen, F., Li, Z., Shen, G., Cui, D., & Chen, X. (2012). Light-triggered theranostics based on photosensitizer-conjugated carbon dots for simultaneous enhanced-fluorescence imaging and photodynamic therapy. Advanced Materials, 24(37). https://doi.org/10.1002/adma.201200650
32. Im, K., Mareninov, S., Diaz, M. F. P., & Yong, W. H. (2019). An introduction to performing immunofluorescence staining. In Methods in Molecular Biology (Vol. 1897). https://doi.org/10.1007/978-1-4939-8935-5_26
33. Immunofluorescence Test—an overview|ScienceDirect Topics. (n.d.). Retrieved Apr. 17, 2025, from https://www.sciencedirect.com/topics/medicine-and-dentistry/immunofluorescence-test
34. Kannoth, S. (2012). Paraneoplastic neurologic syndrome: A practical approach. In Annals of Indian Academy of Neurology (Vol. 15, Issue 1). https://doi.org/10.4103/0972-2327.93267
35. Kazarian, M., & Laird-Offringa, I. A. (2011). Small-cell lung cancer-associated autoantibodies: potential applications to cancer diagnosis, early detection, and therapy. In Molecular Cancer (Vol. 10). https://doi.org/10.1186/1476-4598-10-33
36. Keene, J. D. (2007). RNA regulons: Coordination of post-transcriptional events. In Nature Reviews Genetics (Vol. 8, Issue 7). https://doi.org/10.1038/nrg2111
37. Kurien, B. T., & Hal Scofield, R. (2015). Western blotting: An introduction. In Western Blotting: Methods and Protocols. https://doi.org/10.1007/978-1-4939-2694-7_5
38. Lei, X. X., Liu, Y., Wang, J. X., Cai, Q., Yan, M., He, H. P., Liu, Q., Long, Z. J., & Guan, Z. (2020). SOX1 promotes differentiation of nasopharyngeal carcinoma cells by activating retinoid metabolic pathway. Cell Death and Disease, 11(5). https://doi.org/10.1038/s41419-020-2513-1
39. Li, L., Guo, Y., & Wang, J. (2023). Detection of paraneoplastic antibodies and their significance in paraneoplastic neurologic syndromes: a narrative review. Annals of Translational Medicine, 11(7). https://doi.org/10.21037/atm-21-2434
40. Mahmood, T., & Yang, P. C. (2012). Western blot: Technique, theory, and trouble shooting. North American Journal of Medical Sciences, 4(9). https://doi.org/10.4103/1947-2714.100998
41. Marsili, L., Marcucci, S., LaPorta, J., Chirra, M., Espay, A. J., & Colosimo, C. (2023). Paraneoplastic Neurological Syndromes of the Central Nervous System: Pathophysiology, Diagnosis, and Treatment. In Biomedicines (Vol. 11, Issue 5). https://doi.org/10.3390/biomedicines11051406
42. Matschke, J., Kromminga, A., Erbersdobler, A., Lamszus, K., Anders, S., & Köfüncü, E. (2007). Paraneoplastic cerebellar degeneration and anti-Yo antibodies in a man with prostatic adenocarcinoma [6]. In Journal of Neurology, Neurosurgery and Psychiatry (Vol. 78, Issue 7). https://doi.org/10.1136/jnnp.2006.112961
43. Matsubara, S., Yamaji, Y., Sato, M., Fujita, J., & Takahara, J. (1996). Expression of a photoreceptor protein, recoverin, as a cancer-associated retinopathy autoantigen in human lung cancer cell lines. British Journal of Cancer, 74(9). https://doi.org/10.1038/bjc.1996.558
44. Murinson, B. B., & Guarnaccia, J. B. (2008). Stiff-person syndrome with amphiphysin antibodies: Distinctive features of a rare disease. Neurology, 71(24). https://doi.org/10.1212/01.wnl.0000327342.58936.e0
45. Oh, J. H., & Cho, J. Y. (2023). Comparative oncology: overcoming human cancer through companion animal studies. In Experimental and Molecular Medicine (Vol. 55, Issue 4). https://doi.org/10.1038/s12276-023-00977-3
46. O'Kell, A. L., Shome, M., Qiu, J., Williams, S., Chung, Y., LaBaer, J., Atkinson, M. A., & Wasserfall, C. (2022). Exploration of autoantibody responses in canine diabetes using protein arrays. Scientific Reports, 12(1). https://doi.org/10.1038/s41598-022-06599-5
47. O'Leary, C. G., Battley, J. E., & O'Reilly, S. (2017). Anti-Ri antibody associated small cell lung carcinoma. In Irish Journal of Medical Science (Vol. 186, Issue 2). https://doi.org/10.1007/s11845-016-1402-1
48. Pancotto, T. E., & Rossmeisl, J. H. (2017). A case of stiff dog syndrome associated with anti-glutamic acid decarboxylase antibodies. Journal of Clinical Movement Disorders, 4(1). https://doi.org/10.1186/s40734-017-0053-3
49. Peng, Y., Yang, H., Xue, Y. H., Chen, Q., Jin, H., Liu, S., Yao, S. Y., & Du, M. Q. (2023). An update on malignant tumor-related stiff person syndrome spectrum disorders: clinical mechanism, treatment, and outcomes. In Frontiers in Neurology (Vol. 14). https://doi.org/10.3389/fneur.2023.1209302
50. Ricigliano, V. A. G., Fossati, B., Saraceno, L., Cavalli, M., Bazzigaluppi, E., & Meola, G. (2018). MSA mimic? Rare occurrence of anti-hu autonomic failure and thymoma in a subject with parkinsonism: Case report and literature review. Frontiers in Neuroscience, 12(January). https://doi.org/10.3389/fnins.2018.00017
51. Rohdin, C., Ljungvall, I., Jäderlund, K. H., Svensson, A., Lindblad-Toh, K., & Häggström, J. (2024). Assessment of glial fibrillary acidic protein and anti-glial fibrillary acidic protein autoantibody concentrations and necrotising meningoencephalitis risk genotype in dogs with pug dog myelopathy. Veterinary Record, 194(12). https://doi.org/10.1002/vetr.3895
52. Ruiz-García, R., Martínez-Hernández, E., Saiz, A., Dalmau, J., & Graus, F. (2020). The Diagnostic Value of Onconeural Antibodies Depends on How They Are Tested. Frontiers in Immunology, 11. https://doi.org/10.3389/fimmu.2020.01482
53. Scharf, M., Miske, R., Kade, S., Hahn, S., Denno, Y., Begemann, N., Rochow, N., Radzimski, C., Brakopp, S., Probst, C., Teegen, B., Stöcker, W., & Komorowski, L. (2018). A spectrum of neural autoantigens, newly identified by histo-immunoprecipitation, mass spectrometry, and recombinant cell-based indirect immunofluorescence. Frontiers in Immunology, 9(July). https://doi.org/10.3389/fimmu.2018.01447
54. Schiffman, J. D., & Breen, M. (2015). Comparative oncology: What dogs and other species can teach us about humans with cancer. In Philosophical Transactions of the Royal Society B: Biological Sciences (Vol. 370, Issue 1673). https://doi.org/10.1098/rstb.2014.0231
55. Shurin, M. R., & Wheeler, S. E. (2024). Clinical Significance of Uncommon, Non-Clinical, and Novel Autoantibodies. https://doi.org/10.2147/ITT.S450184
56. Sormunen, A., Koivulehto, E., Alitalo, K., Saksela, K., Laham-Karam, N., & Ylä-Herttuala, S. (2023). Comparison of Automated and Traditional Western Blotting Methods. Methods and Protocols, 6(2). https://doi.org/10.3390/mps6020043
57. Stafford, E. G., Kortum, A., Castel, A., Green, L., Lau, J., Early, P. J., Muñana, K. R., Mariani, C. L., Yoder, J. A., & Olby, N. J. (2019). Presence of cerebrospinal fluid antibodies associated with autoimmune encephalitis of humans in dogs with neurologic disease. Journal of Veterinary Internal Medicine, 33(5). https://doi.org/10.1111/jvim.15616
58. Tan, F., Thiele, C. J., & Li, Z. (2014). Collapsin response mediator proteins: Potential diagnostic and prognostic biomarkers in cancers (review). In Oncology Letters (Vol. 7, Issue 5). https://doi.org/10.3892/ol.2014.1909
59. Tawa, G. J., Braisted, J., Gerhold, D., Grewal, G., Mazcko, C., Breen, M., Sittampalam, G., & LeBlanc, A. K. (2021). Transcriptomic profiling in canines and humans reveals cancer specific gene modules and biological mechanisms common to both species. PLoS Computational Biology, 17(9). https://doi.org/10.1371/journal.pcbi.1009450
60. van Beek, N., Krüger, S., Fuhrmann, T., Lemcke, S., Goletz, S., Probst, C., Komorowski, L., Di Zenzo, G., Dmochowski, M., Drenovska, K., Horn, M., Jedlickova, H., Kowalewski, C., Medenica, L., Murrell, D., Patsatsi, A., Geller, S., Uzun, S., Vassileva, S., . . . Rentzsch, K. (2020). Multicenter prospective study on multivariant diagnostics of autoimmune bullous dermatoses using the BIOCHIP technology. Journal of the American Academy of Dermatology, 83(5). https://doi.org/10.1016/j.jaad.2020.01.049
61. Wang, Z., Li, Z., Wu, Q., Li, C., Li, J., Zhang, Y., Wang, C., Sun, S., & Sun, S. (2020). DNER promotes epithelial-mesenchymal transition and prevents chemosensitivity through the Wnt/β-catenin pathway in breast cancer. Cell Death and Disease, 11(8). https://doi.org/10.1038/s41419-020-02903-1
62. Wei, H., Ren, K., Zhang, Q., Jin, Y., Cao, B., Tian, Z., Mao, T., & Ren, L. (2023). Titin as a potential novel therapeutic target in colorectal cancer. Journal of Cellular and Molecular Medicine, 27(19). https://doi.org/10.1111/jcmm.17866
63. Wisdom, G. B. (2005). Conjugation of antibodies to alkaline phosphatase. Methods in Molecular Biology (Clifton, N.J.), 295. https://doi.org/10.1385/1-59259-873-0:123
64. Yu, Z., Kryzer, T. J., Griesmann, G. E., Kim, K K., Benarroch, E. E., & Lennon, V. A. (2001). CRMP-5 neuronal autoantibody: Marker of lung cancer and thymoma-related autoimmunity. Annals of Neurology, 49(2). https://doi.org/10.1002/1531-8249(20010201)49:2<146::AID-ANA34>3.0.CO;2-E

What is claimed is:

1. A method for detecting cancer in an animal, including at least one of a dog, a cat, a rabbit, or a ferret, comprising:
(a) retrieving a blood specimen from the animal,
(b) adding a buffer to the specimen to form a composite specimen,
(c) binding the composite specimen to at least one antigen selected from a group of intracellular antigens consisting of amphiphysin, CV2, PNMa2/Ta, Ri, Yo, Hu, recoverin, SOX1, titin, zic4, GAD65, and Tr (DNER), wherein the at least one antigen of the group of intracellular antigens is immobilized on a test strip, and
(d) optically detecting a presence of at least one antigen-antibody binding complex using a predetermined threshold.

2. The method, as recited in claim 1, further comprising:
providing an indication of positive for cancer when the presence of at least one antigen-antibody binding complex exceeds the predetermined threshold.

3. The method, as recited in claim 1, wherein step (d) is done using an optical scanner.

4. The method, as recited in claim 1, wherein step (d) is done using a microscope.

5. The method, as recited in claim 1, further comprising a step of providing an indication of positive for cancer associated with at least one of lung cancer, mammary tumor, thymoma, lymphoma, or intestinal tumor when the antigen-antibody binding complex detected in step (d) is associated with amphiphysin.

6. The method, as recited in claim 1, further comprising a step of providing an indication of positive for cancer associated with at least one of lung cancer, thymoma, lymphoma, uterine tumor, prostate cancer, kidney cancer, intestinal tumor, mammary tumor, thyroid cancer, and squamous cell carcinoma when the antigen-antibody binding complex detected in step (d) is associated with CV2.

7. The method, as recited in claim 1, further comprising a step of providing an indication of positive for cancer associated with at least one of testicular tumor, lung cancer, salivary gland adenocarcinoma, mammary tumor, ovarian tumor, intestinal tumor, kidney tumor, and lymphoma when the antigen-antibody binding complex detected in step (d) is associated with PNMa2/Ta.

8. The method, as recited in claim 1, further comprising a step of providing an indication of positive for cancer associated with at least one of lung cancer, mammary tumor, lymphoma, brain tumor, bladder cancer, ovarian tumor or testicular tumor when the antigen-antibody binding complex detected in step (d) is associated with Ri.

9. The method, as recited in claim 1, further comprising a step of providing an indication of positive for cancer associated with at least one of ovarian tumor, mammary tumor, uterine tumor, esophageal cancer, prostate cancer, gallbladder tumor, bladder cancer, lymphoma, thymoma and melanoma when the antigen-antibody binding complex detected in step (d) is associated with Yo.

10. The method, as recited in claim 1, further comprising a step of providing an indication of positive for cancer associated with at least one of lung cancer, neuroblastoma, prostate cancer, bladder tumor, ovarian tumor, mammary tumor, pancreatic cancer and intestinal tumor when the antigen-antibody binding complex detected in step (d) is associated with Hu.

11. The method, as recited in claim 1, further comprising a step of providing an indication of positive for cancer associated with at least one of lung cancer, melanoma, mammary tumor, ovarian tumor, uterine tumor, intestinal tumor, kidney cancer, pancreatic cancer, prostate cancer, lymphoma, basal cells and squamous cell carcinoma when the antigen-antibody binding complex detected in step (d) is associated with recoverin.

12. The method, as recited in claim 1, further comprising a step of providing an indication of positive for cancer associated with at least one of lung cancer or thyroid cancer when the antigen-antibody binding complex detected in step (d) is associated with SOX1.

13. The method, as recited in claim 1, further comprising a step of providing an indication of positive for cancer associated with thymoma when the antigen-antibody binding complex detected in step (d) is associated with titin.

14. The method, as recited in claim 1, further comprising a step of providing an indication of positive for cancer associated with at least one of lung cancer, ovarian tumor, mammary tumor, thyroid cancer, and lymphoma when the antigen-antibody binding complex detected in step (d) is associated with zic4.

15. The method, as recited in claim 1, further comprising a step of providing an indication of positive for cancer associated with at least one of lung cancer, thymoma, mammary tumor, intestinal tumor, kidney cancer, lymphoma, uterine tumor, pancreatic cancer, multiple myeloma or testicular tumor when the antigen-antibody binding complex detected in step (d) is associated with GAD65.

16. The method, as recited in claim 1, further comprising a step of providing an indication of positive for cancer associated with at least one of mammary tumor, lymphoma, lung cancer, uterine tumor or brain tumor when the antigen-antibody binding complex detected in step (d) is associated with Tr (DNER).

17. A method for detecting cancer in a mammalian animal, comprising:
(a) providing a nitrocellulose test strip with an antigen-containing solid phase coated with at least one immobilized recombinant antigen selected from a group of intracellular antigens consisting of amphiphysin, CV2, PNMa2/Ta, Ri, Yo, Hu, recoverin, SOX1, titin, zic4, GAD65, and Tr (DNER),
(b) retrieving a blood specimen from the animal,
(c) adding a buffer to the specimen to form a composite specimen,
(d) exposing the composite specimen to the antigen-containing solid phase of the test strip to allow for at least one antigen-antibody binding complex to form,
(e) detecting the at least one antibody concentration by optically measuring a color intensity of the at least one antigen-antibody binding complex, and
(f) providing an indication of positive for cancer if the concentration of the at least one antibody in step (e) exceeds a predetermined concentration threshold.
